(19) United States
(12) Patent Application Publication
(10) Pub. No.: US 2005/0120392 A1
(43) Pub. Date: Jun. 2, 2005

(54) Transgenic zebrafish models for thrombosis

(76) Inventors: Amy L. Rubinstein, Atlanta, GA (US); Shuo Lin, Los Angeles, CA (US); Thanh Doan, Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 10/505,922

(22) PCT Filed: Feb. 28, 2003
(86) PCT No.: PCT/US03/06354

Related U.S. Application Data
(60) Provisional application No. 60/360,711, filed on Feb. 28, 2002.

(51) Int. Cl. A01K 67/027

(52) U.S. Cl. 800/3; 800/20

(57) ABSTRACT

The present invention relates to zebrafish models of thrombosis that allow screening of compounds for anti-thrombotic or thrombotic properties in vivo in a whole vertebrate organism. The present invention also relates to the identification and validation of platelet genes as targets for anti-thrombotic or thrombotic compounds.

This application claims priority to U.S. provisional application Ser. No. 60/360,711, filed Feb. 28, 2002, which is herein incorporated by this reference in its entirety.

### FIELD OF THE INVENTION

The present invention relates to zebrafish models of thrombosis that allow screening of compounds for anti-thrombotic or thrombotic properties in vivo in a whole vertebrate organism. The present invention also relates to the identification and validation of platelet genes as targets for anti-thrombotic or thrombotic compounds.

### BACKGROUND

Cardiovascular disease, often the result of thrombotic complications, is one of the leading causes of death in the United States and worldwide. While an enormous medical need exists for novel, innovative thrombosis drugs, only a limited number of targets have been identified and screened to date. Identification of novel targets is critical to the development of new and more effective therapies. The zebrafish represents a unique and untapped resource for identifying and validating such targets.

Since human platelets do not have nuclei it is difficult to take a genomics approach to target identification in humans. Zebrafish platelets contain nuclei with a full complement of DNA, actively transcribing genes that can be used to identify platelet relevant or platelet-specific transcripts that may be useful as targets for the development of anti-thrombotic drugs. While it is not known whether there are separate platelet precursor cells in zebrafish that play a role similar to megakaryocytes in humans, there is ample evidence of overlapping function between human and zebrafish platelets.

The high degree of overall conservation between the human and zebrafish genomes and the high level of conservation of the biology of thrombosis suggests that the differences in platelet structure can be effectively exploited for the discovery of relevant anti-thrombosis targets in humans. For example, the zebrafish genome is approximately 75% similar to the human genome and several genes that are known to play a role in thrombosis have already been identified in zebrafish by sequence homology (Jagadeeswaran, et al., 1999; Sheehan, et al., 2001). More importantly, several in vitro assays have established that zebrafish blood responds to clotting agents such as collagen and anti-thrombotics such as aspirin and warfarin in a manner similar to human blood (Jagadeeswaran and Liu, 1997; Jagadeeswaran and Sheehan, 1998; Jagadeeswaran, et al., 1999). Finally, zebrafish mutants have been identified that exhibit the same characteristics as certain human hematological diseases (Brownlie, et al., 1998; Childs, et al., 2000; Donovan, et al., 2000; Liao, et al., 2000; Wang, et al., 1998).

New thrombotic drug candidates are often tested in a number of in vitro assays that measure such aspects of thrombosis as platelet aggregation and adhesion. Instead of choosing one aspect, such as cell adhesion and studying it in isolation, zebrafish assays enable observation of the integrated process in vivo.

Zebrafish are vertebrates that develop rapidly outside of the mother and are transparent during development so one can observe platelet formation and clotting events in vivo and in real-time. Several animal models presently exist for the evaluation of new thrombotic drugs (Hermann, 1983; Sato and Ohshima, 1984; Leadley, et al., 2000). However, these animal models are not suited for screening large numbers of compounds. Zebrafish embryos can be used to screen compounds in 50-100 microliter volumes. Test compounds have been shown to have reproducible effects. Zebrafish lay 200-400 eggs a week so large numbers of different fish lines can be tested rapidly for drug efficacy and toxicity, providing a valuable secondary screening capability for targeted libraries or hits from primary screens, as well as compound profiling.

This invention provides a zebrafish model of thrombosis that utilizes transgenic zebrafish with fluorescent platelets in order to screen for agents or compounds that are anti-thrombotic or thrombotic. This zebrafish model is useful for target identification and validation, as well as for compound profiling and drug screening.

### SUMMARY OF THE INVENTION

The present invention provides a method of identifying an anti-thrombotic compound comprising: a) contacting a transgenic zebrafish containing aggregated platelets that express a reporter protein, with a test compound; b) comparing the platelets in the zebrafish contacted with the test compound with the platelets of a transgenic zebrafish that was not contacted with the test compound; and c) determining the effect of the test compound on platelet aggregation, such that if platelet aggregation in the zebrafish contacted with the test compound is less than platelet aggregation in the zebrafish that was not contacted with the test compound, the compound is an anti-thrombotic compound.

Also provided by the present invention is a method of identifying a compound that prevents thrombosis comprising: a) contacting a transgenic zebrafish expressing a reporter protein in platelets, with a thrombotic compound and test compound; b) comparing the platelets in the zebrafish contacted with the thrombotic compound and the test compound with the platelets of a transgenic zebrafish that was contacted only with the thrombotic compound; c) determining the effect of the test compound on the platelets, such that if platelet aggregation in the zebrafish contacted with the thrombotic compound and the test compound is less than platelet aggregation in the zebrafish that was contacted only with the thrombotic compound, the compound prevents thrombosis.

The present invention provides a method of identifying a thrombotic compound comprising: a) contacting a transgenic zebrafish containing platelets that express a reporter protein, with a test compound; b) comparing the platelets in the transgenic zebrafish contacted with the test compound with the platelets of a transgenic zebrafish that was not contacted with the test compound; and c) determining the effect of the test compound on platelet aggregation, such that if platelet aggregation in the zebrafish contacted with the test compound is greater than platelet aggregation in the zebrafish that was not contacted with the test compound, the compound is a thrombotic compound.

The present invention also provides a method of identifying a platelet gene that is involved in platelet function comprising: a) comparing a transgenic zebrafish containing platelets that express a reporter protein, with a transgenic zebrafish containing platelets that express a reporter protein and has a platelet gene knocked out; and b) determining the effect of the platelet gene knockout on platelet function such that if there is a difference between the platelets of the transgenic zebrafish containing platelets that express a reporter protein and the transgenic zebrafish containing platelets that express a reporter protein and has a platelet gene knockout, the platelet gene is involved in platelet function.

Also provided by the present invention is a method of identifying a platelet gene as a target for an anti-thrombotic compound comprising: a) contacting a transgenic zebrafish containing aggregated platelets that express a reporter protein with an anti-thrombotic compound; b) contacting a transgenic zebrafish containing aggregated platelets that express a reporter protein and has a platelet gene knocked out with an anti-thrombotic compound; c) comparing the platelets in the transgenic zebrafish containing aggregated platelets that express a reporter protein with the platelets in the transgenic zebrafish containing aggregated platelets that express a reporter protein and has a platelet gene knocked out; and d) determining the effect of the anti-thrombotic compound on platelet aggregation, such that if platelet aggregation in the transgenic zebrafish containing aggregated platelets that express a reporter protein is less than platelet aggregation in the zebrafish containing aggregated platelets that express a reporter protein and has a platelet gene knocked out, the platelet gene is a target for the anti-thrombotic compound.

The present invention also provides a method of identifying a platelet gene as a target for a thrombotic compound comprising: a) contacting a transgenic zebrafish containing platelets that express a reporter protein with a thrombotic compound; b) contacting a transgenic zebrafish containing platelets that express a reporter protein and has a platelet gene knocked out with a thrombotic compound; c) comparing the platelets in the transgenic zebrafish containing aggregated platelets that express a reporter protein with the platelets in the transgenic zebrafish containing aggregated platelets that express a reporter protein and has a platelet gene knocked out; and d) determining the effect of the thrombotic compound on platelet aggregation, such that if platelet aggregation in the transgenic zebrafish containing platelets that express a reporter protein is greater than platelet aggregation in the zebrafish containing platelets that express a reporter protein and has a platelet gene knocked out, the platelet gene is a target for the thrombotic compound.

The present invention also provides a method of identifying an anti-thrombotic compound that affects platelet aggregation via a platelet gene comprising: a) contacting a transgenic zebrafish containing aggregated platelets that express a reporter protein with a test compound; b) contacting a transgenic zebrafish containing aggregated platelets that express a reporter protein and has a platelet gene knocked out with a test compound; c) comparing the platelets in the transgenic zebrafish containing aggregated platelets that express a reporter protein with the platelets in the transgenic zebrafish containing aggregated platelets that express a reporter protein and has a. platelet gene knocked out; and d) determining the effect of the test compound on platelet aggregation, such that if platelet aggregation in the transgenic zebrafish containing aggregated platelets that express a reporter protein is less than platelet aggregation in the zebrafish containing aggregated platelets that express a reporter protein and has a platelet gene knocked out, the compound is an anti-thrombotic compound that affects platelet aggregation via the platelet gene that has been knocked out.

Also provided by the present invention is a method of identifying a thrombotic compound that affects platelet aggregation via a platelet gene comprising:a) contacting a transgenic zebrafish containing platelets that express a reporter protein with a test compound; b) contacting a transgenic zebrafish containing platelets that express a reporter protein and has a platelet gene knocked out with a test compound; c) comparing the platelets in the transgenic zebrafish containing aggregated platelets that express a reporter protein with the platelets in the transgenic zebrafish containing aggregated platelets that express a reporter protein and has a platelet gene knocked out; and d) determining the effect of the test compound on platelet aggregation, such that if platelet aggregation in the transgenic zebrafish containing platelets that express a reporter protein is greater than platelet aggregation in the zebrafish containing platelets that express a reporter protein and has a platelet gene knocked out, the compound is a thrombotic compound that affects platelet aggregation via the platelet gene that has been knocked out.

Also provided by the present invention is a method of identifying a platelet gene that is involved in platelet function comprising: a) comparing the platelets in a transgenic zebrafish containing platelets that express a reporter protein, with the platelets in a transgenic zebrafish containing platelets that express a reporter protein and overexpress the product of the platelet gene; and b) determining the effect of the overexpression of the product of the platelet gene on platelet function such that if there is a difference between the platelets of the transgenic zebrafish containing platelets that express a reporter protein and the transgenic zebrafish containing platelets that express a reporter protein and has a platelet gene overexpressed, the platelet gene is involved in platelet function.

Further provided by the present invention is a method of identifying a known compound with a known target, as a compound that prevents thrombosis comprising: a) contacting a transgenic zebrafish expressing a reporter protein in platelets, with a thrombotic compound and the known compound; b) comparing the platelets in the zebrafish contacted with the thrombotic compound and the known compound with the platelets of a transgenic zebrafish that was contacted only with the thrombotic compound; and c) determining the effect of the known compound on the platelets, such that if platelet aggregation in the zebrafish contacted with the thrombotic compound and the known compound is less than platelet aggregation in the zebrafish that was contacted only with the thrombotic compound, the known compound is a compound that prevents thrombosis via its known target.

Also provided by the present invention is a method of identifying a known compound with a known target, as a thrombotic compound comprising: a) contacting a transgenic zebrafish containing platelets that express a reporter protein, with a known compound; b) comparing the platelets in the zebrafish contacted with the known compound with the platelets of a transgenic zebrafish that was not contacted with the known compound; and c) determining the effect of the test compound on platelet aggregation, such that if platelet aggregation in the zebrafish contacted with the known compound is greater than platelet aggregation in the zebrafish that was not contacted with the test compound, the known compound is a thrombotic compound that affects platelet aggregation via its known target.

Also provided is a method of identifying a target for an anti-thrombotic compound comprising: a) contacting a transgenic zebrafish expressing a reporter protein in platelets, with a thrombotic compound and a known compound for which a target is known; b) comparing the platelets in the zebrafish contacted with the thrombotic compound and the known compound with the platelets of a transgenic zebrafish that was contacted only with the thrombotic compound; and c) determining the effect of the known compound on the platelets, such that if platelet aggregation in the zebrafish contacted with the thrombotic compound and the known compound is less than platelet aggregation in the zebrafish that was contacted only with the thrombotic compound, the known compound is a compound that prevents thrombosis via its known target, thus identifying a target for an anti-thrombotic compound.

A method of identifying a target for a thrombotic compound comprising: a) contacting a transgenic zebrafish containing platelets that express a reporter protein, with a known compound for which a target is known; b) comparing the platelets in the zebrafish contacted with the known compound with the platelets of a transgenic zebrafish that was not contacted with the known compound; and c) determining the effect of the test compound on platelet aggregation, such that if platelet aggregation in the zebrafish contacted with the known compound is greater than platelet aggregation in the zebrafish that was not contacted with the test compound, the known compound is a thrombotic compound that affects platelet aggregation via its known target, thus identifying a target for a thrombotic compound.

### BRIEF DESCRIPTION OF THE FIGURES

FIG. 1 shows fluorescent images of platelets in TG(GPIIb:eGFP) zebrafish. The yellow arrows indicate platelets that were not moving, while the red arrows show platelets that were in motion.

FIG. 2 is a bright field image of a 7 dpf larva with illustration of the sections used for scoring in the assay. All data represented are expressed as means±standard error. Statistical significance was determined when p<0.05 using student's unpaired, two-tailed distribution with unequal variance t-test for all samples.

FIG. 3 shows that acetylsalicylic acid (aspirin) prevented the ADP-induced microaggregate formation in GPIIb/eGFP zebrafish. 5 dpf larvae were soaked. overnight with varying concentration of aspirin and then were challenged with 90 pmol of ADP the following day. No statistical difference was observed within the aspirin-treated group. Statistical significance for ADP-induced microaggregate formation was determined for each aspirin-treated group with respect to ADP alone.

FIG. 4 illustrates that Abciximab (Reopro) inhibited the in vivo ADP-induced microaggregate formation of platelets. Larvae (5 dpf) were soaked overnight with varying concentrations of Reopro. The following day, Reopro (120 ng) was injected into the larvae before the challenge with 90 pmol of ADP. Baseline value for phenol red injections was taken into account for the data shown. Statistical significance was achieved at 0.5-5 μg/ml of Reopro treatment.

FIG. 5 shows Eptifibatide (Integrilin) dose-dependently prevented ADP-induced aggregate formation in TG(GPIIb:eGFP) zebrafish. Five days post-fertilized larvae were soaked overnight with varying concentration of Integrilin and then were challenged with 90 pmol of ADP the following day. Statistical significance for ADP-induced microaggregate formation was determined for each Integrilin-treated group with respect to ADP alone.

FIG. 6 shows Ticlopidine dose-dependently prevented ADP-induced aggregate formation in TG(GPIIb:eGFP) zebrafish. Larvae (5 dpf) were soaked overnight with varying concentration of Ticlopidine and then were challenged with 90 pmol of ADP the following day. Statistical significance for ADP-induced microaggregate formation was determined for each Ticlopidine-treated group with respect to ADP alone.

FIG. 7 shows that hirudin inhibited the thrombin-induced aggregate formation in TG(GPIIb:eGFP) zebrafish. Larvae (6-7 dpf) were challenged with 0.018 NIH unit of thrombin or 90 pmol of ADP, either in the presence or absence of 0.029 unit of hirudin. Statistical significance for aggregate formation was determined for the hirudin group with respect to its agonist.

FIG. 8 is a FACs analysis of cells isolated from 6-7 dpf TG(GPIIb:eGFP) zebrafish. Cells from wild-type (A), heterozygous (B) or homozygous (C) 6-7 dpf TG(GPIIb:eGFP) zebrafish were isolated and sorted with the flow cytometer using a channel for GFP. D, Cell suspension containing propidium iodide was sorted using propidium iodide channel to determine cell viability. E, Cells collected after the first GFP-sort were reanalyzed through the flow cytometer. F, Cells collected after the first GFP-sort were analyzed for propidium iodide staining.

FIG. 9 shows the detection of eGFP mRNA in 4 dpf and 8 dpf TG(GPIIb:eGFP) zebrafish using in situ hybridization. Larvae at 4 dpf (left panel, arrows show heavy staining in the intermediate cell mass) and 8 dpf (right panel, arrow points are the pronephros region) were fixed with paraformaldehyde. In situ hybridization was performed using a 750 bp riboprobe for eGFP. The images were representative of 4 larvae for each age group.

FIG. 10 shows that morpholinos recognizing the GPIIb or P2Y₁₂ mRNA, were effective in reducing in vivo ADP-induced aggregation in zebrafish. Morpholinos were injected into fertilized eggs at the 1-4 cell stage. At 5-6 days after morpholino injection, 90 pmol of ADP was injected into the heart cavity of the larvae. Using a fluorescent microscope, the presence or absence of moving platelets was determined. The percent of larvae that showed a complete inhibition of platelet movement is shown. Mock injection (for no morpholino) is shown as 0 in the figure. n represents the number of larvae tested for each given condition.

### DETAILED DESCRIPTION OF THE INVENTION

The present invention may be understood more readily by reference to the following detailed description of the preferred embodiments of the invention and the Example included therein.

Before the present compounds and methods are disclosed and described, it is to be understood that this invention is not limited to specific proteins or specific methods. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise.

The present invention is more particularly described in the following examples which are intended as illustrative only since numerous modifications and variations therein will be apparent to those skilled in the art.

The present invention provides a method of identifying an anti-thrombotic compound comprising: a) contacting a transgenic zebrafish containing aggregated platelets that express a reporter protein, with a test compound; b) comparing the platelets in the transgenic zebrafish contacted with the test compound with the platelets of a transgenic zebrafish that was not contacted with the test compound; and c) determining the effect of the test compound on platelet aggregation, such that if platelet aggregation in the zebrafish contacted with the test compound is less than platelet aggregation in the zebrafish that was not contacted with the test compound, the compound is an anti-thrombotic compound.

As utilized herein, a “compound” can be but is not limited to a chemical, a small molecule, a drug, an antibody, a peptide, a secreted protein, a nucleic acid (such as DNA, RNA, a polynucleotide, an oligonucleotide or a cDNA) or an antisense molecule.

The transgenic zebrafish of this invention can be a transient or a stable transgenic zebrafish. The transgenic zebrafish of this invention include zebrafish larvae, zebrafish embryos and adult zebrafish. The transgenic zebrafish in which the expression of a reporter protein is tissue-specific is contemplated for this invention. For example, transgenic animals that express a reporter protein at specific sites such as megakaryocytes or platelets can be produced by introducing a nucleic acid into fertilized eggs, embryonic stem cells or the germline of the animal, wherein the nucleic acid is under the control of a specific promoter which allows expression of the nucleic acid in specific types of cells (e.g., a promoter which allows expression only in platelets). As used herein, a protein or gene is expressed predominantly in a given tissue, cell type, cell lineage or cell, when 90% or greater of the observed expression occurs in the given tissue cell type, cell lineage or cell.

More specifically, this invention contemplates the use of a transgenic zebrafish that expresses a reporter protein that is under the control of a platelet-specific promoter such as, but not limited to, a platelet receptor glycoprotein IIb promoter, a glycoprotein VI promoter, a platelet ADP receptor P2Y12 promoter, a P₂Y1 promoter, a protease-activated receptor-1, -2, -3, -4 promoter, a glycoprotein V promoter, a glycoprotein IX promoter, a glycoprotein Ib alpha promoter, a glycoprotein Ib beta promoter, a platelet factor IV promoter, a platelet basic protein promoter or a thrombopoietin receptor promoter, and is expressed in platelets.

The expression sequences used to drive expression of the reporter proteins can be isolated by one of skill in the art, for example, by screening a genomic zebrafish library for sequences upstream of the zebrafish gene of interest. The expression sequences can include a promoter, an enhancer, a silencer and necessary information processing sites, such as ribosome binding sites, RNA splice sites, polyadenylation sites and transcriptional terminator sequences.

By utilizing a transgenic zebrafish that expresses a fluorescent protein under the control of a platelet-specific promoter, the platelets can be visualized in the developing embryo and in later states of zebrafish development. Thus, if a zebrafish of the present invention is exposed to a thrombotic compound or any other compound causing platelet aggregation, such aggregation should be readily apparent by monitoring fluorescence. Zebrafish embryos can be easily “cultured” in 96 well plates where they can be soaked in test compounds. The effects of the test compound can also be readily visualized. If the test compound reduces platelet aggregation, one of skill in the art will be able to visualize the disaggregation of platelets by observing the pattern of fluorescence in the zebrafish platelets. For example, if prior to administering a test compound, the skilled artisan observes a fluorescent mass, or clot of platelets and after administration of the test compound the size of the mass is reduced or increased circulation of fluorescent platelets is observed at the site, the test compound reduces platelet aggregation. Reduction of platelet aggregation does not have to be complete as the efficacy of the test compound can range from a slight reduction in platelet aggregation to complete dissolution of a platelet aggregate or clot.

The anti-thrombotic compounds identified by the methods of the present invention can be utilized to treat disease states associate with thrombosis. As used herein, “thrombosis” is the process of intravascular formation of a blood clot comprised of fibrin and platelets. Disease states associated with thrombosis include, but are not limited to, myocardial infarction, atherosclerosis, restenosis after stent or angioplasty, acute renal allograft rejection, stroke, coronary artery disease, deep vein thrombosis, thrombosis of sickle cell anemia, unstable angina. The anti-thrombotic compounds identified by the methods of the present invention can also be utilized in other in vitro assays, such as cell adhesion assays, or platelet aggregation assays to study the effects of the compounds on human platelets. Furthermore, the anti-thrombotic compounds can be utilized in other in vivo animal models of thrombosis or other disease states associated with thrombosis, such as a mouse model, a rat model, a rabbit model or a baboon model of thrombosis to study their therapeutic effects.

Once anti-thrombotic or thrombotic compounds are identified that can be useful for the treatment of disease, these compositions can be used therapeutically in combination with a pharmaceutically acceptable carrier. By “pharmaceutically acceptable carrier” is meant a material that is not biologically or otherwise undesirable, that is, the material may be administered to an individual along with a polypeptide, nucleic acid, or other compound of the invention without causing any undesirable biological effects or interacting in a deleterious manner with any of the components of the pharmaceutical composition in which it is contained. Pharmaceutical carriers are well-known in the art. These most typically are standard carriers for administration of vaccines or pharmaceuticals to humans, including solutions such as sterile water, saline, and buffered solutions at physiological pH.

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like. Methods for making such formulations are well known in the art, and are described, for example, in: Remington: The Science and Practice of Pharmacy (19^{th} ed.), ed. A. R. Gennaro, E. W. Martin Mack Publishing Co., Easton, Pa., 1995.

The pharmaceutical compositions may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The compounds and compositions of the present invention can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids or binders may be desirable. Formulations for parenteral administration may include sterile aqueous solutions which may also contain buffers, diluents and other suitable additives.

The compounds of the invention are administered in an effective amount, using standard approaches. Effective dosages and schedules for administering the compounds may be determined empirically, and making such determinations is routine to one of ordinary skill in the art. The skilled artisan will understand that the dosage will vary, depending upon, for example, the species of the subject, the route of administration, the particular compound to be used, other drugs being administered, and the age, condition, sex and extent of the disease in the subject. The dosage can be adjusted by the individual physician in the event of any counterindications. A dose of a compound of the invention generally will range between about 1 μg/kg of body weight and 1 g/kg of body weight. Examples of such dosage ranges are, e.g., about 1 μg-100 μg/kg, 100 μg/kg-10 mg/kg, or 10 mg-1 g/kg, once a week, bi-weekly, daily, or two to four times daily.

The transgenic fish utilized in the methods of this invention are produced by introducing a transgenic construct into cells of a zebrafish, preferably embryonic cells, and most preferably in a single cell embryo, essentially as described in Meng et al. (1998). The transgenic construct is preferably integrated into the genome of the zebrafish, however, the construct can also be constructed as an artificial chromosome. The transgenic construct can be introduced into embryonic cells using any technique known in the art. For example, microinjection, electroporation, liposomal delivery and particle gun bombardment can all be utilized to effect transgenic construct delivery to embryonic cells. Embryos can be microinjected at the one or two cell stage or the construct can be incorporated into embryonic stem cells which can later be incorporated into a growing embryo. Other methods for achieving zebrafish transgenesis that are developed can also be utilized to introduce a construct into an embryo or embryonic stem cells.

Embryos or embryonic cells can be obtained as described in the Examples provided herein. Zebrafish containing a transgene can be identified by numerous methods such as probing the genome of the zebrafish for the presence of the transgene construct by Northern or Southern blotting. Polymerase chain reaction techniques can also be employed to detect the presence of the transgene. Expression of the reporter protein can also be detected by methods known in the art. For example, RNA can be detected using any of numerous nucleic acid detection techniques. Alternatively, an antibody can be used to detect the expression product or one skilled in the art can visualize and quantitate expression of a fluorescent reporter protein such as GFP.

As used herein, a reporter protein is any protein that can be specifically detected when expressed. Reporter proteins are useful for detecting or quantitating expression from expression sequences. For example, operatively linking nucleotide sequences encoding a reporter protein to a tissue specific expression sequence allows one to study lineage development. In such studies, the reporter protein serves as a marker for monitoring developmental processes. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), enhanced green fluorescent protein (eGFP), reef coral fluorescent protein (RCFP), cyan fluorescent protein (CFP), red fluorescent protein (RFP) and yellow fluorescent protein (YFP). For example, by utilizing GFP or RCFP, fluorescence is observed upon exposure to ultraviolet, mercury, xenon, argon or krypton arc light without the addition of a substrate. The use of reporter proteins that, like GFP, are directly detectable without requiring the addition of exogenous factors are preferred for detecting or assessing gene expression during zebrafish development. A transgenic zebrafish embryo, carrying a construct encoding a reporter protein and a tissue-specific expression sequence, such as an expression sequence that directs expression in platelets provides a rapid, real time in vivo system for analyzing spatial and temporal expression patterns of platelets and their interactions.

Also provided by the present invention is a method of identifying a compound that prevents thrombosis comprising: a) contacting a transgenic zebrafish expressing a reporter protein in platelets, with a thrombotic compound and test compound; b) comparing the platelets in the zebrafish contacted with the thrombotic compound and the test compound with the platelets of a transgenic zebrafish that was contacted only with the thrombotic compound; c) determining the effect of the test compound on the platelets, such that if platelet aggregation in the zebrafish contacted with the thrombotic compound and the test compound is less than platelet aggregation in the zebrafish that was contacted only with the thrombotic compound, the compound prevents thrombosis.

For example, one skilled in the art would select transgenic zebrafish embryos or larvae that express a reporter protein in platelets as described in the Examples. If the reporter protein is a fluorescent reporter protein, the skilled artisan will see the fluorescent reporter protein expressed in platelets. In order to assess the preventive properties of a test compound, one would contact the zebrafish with the test compound prior to addition of a thrombotic compound, contact the zebrafish with the test compound and a thrombotic compound concurrently or contact the zebrafish with the test compound after addition of the thrombotic compound. The effects of the test compound are assessed by observing detectable spatial and temporal changes in fluorescence, in situ hybridization signal, or immunohistochemical signal. In the absence of the test compound, the thrombotic compound effects changes in platelets that can be measured both qualitatively and quantitatively. Therefore, an increase in fluorescence at a particular site is observed after addition of a thrombotic compound, i.e. increased fluorescence as a result of platelet aggregation. Thus, if a test compound is effective in preventing thrombosis, upon comparison with a zebrafish exposed only to a thrombotic compound, a change in localized fluorescence should be observed in the zebrafish contacted with both the test compound and the thrombotic compound. Other changes that one of skill in the art would observe include a decrease in clot size in zebrafish that were pre-treated with an anti-thrombotic compound, or dissolution of a clot formed prior to anti-thrombotic compound application. In addition, the rate of platelet circulation could be monitored qualitatively by observation of zebrafish under a fluorescent microscope. Adhesion of platelets to blood vessel walls, in response to thrombotic compound application or blood vessel injury, could also be observed. In the methods of the present invention, the transgenic zebrafish that is exposed only to the thrombotic compound is also a transgenic zebrafish expressing a reporter protein in platelets.

The thrombotic compounds that can be utilized in the methods of this invention to effect platelet adhesion or aggregation, blood clotting or thrombosis include, but are not limited to, collagen, thrombin, ristocetin, arachidonic acid, ADP, platelet activating factor, thromboxanes, prostaglandins, vasopressin, serotonin, and adrenaline. Prior to contacting a zebrafish of the present invention with a thrombotic compound, the zebrafish can be contacted with known anti-thrombotics such as warfarin, aspirin, heparin (including low molecular weight heparins such as Lovenox), GPIIIa/IIb inhibitors (e.g., ReoPro, Aggrastat, Integrilin), and tissue plasminogen activators (e.g., Activase, Retavase), ADP receptor antagonist (e.g., Plavix, Ticlopidine).

In the methods of the present invention, zebrafish can be contacted with a test compound or an anti-thrombotic compound by soaking the zebrafish in the test compound or the anti-thrombotic compound for about 0.25, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 hours prior to contacting the zebrafish with a thrombotic compound. A zebrafish of the present invention which has been soaked in a test compound or an anti-thrombotic compound can also be injected with the test compound or the anti-thrombotic compound prior to contacting the zebrafish with a thrombotic compound. The zebrafish of the present invention can also be contacted with a test compound or an anti-thrombotic by soaking the zebrafish in the test compound or an anti-thrombotic compound after contacting the zebrafish with a thrombotic compound. The zebrafish can also be contacted with a test compound and a thrombotic compound simultaneously, either by soaking the zebrafish in both the test compound and the thrombotic compound, by injecting the zebrafish with the test compound and the thrombotic compound or by utilizing a combination of soaking and injecting the zebrafish with the test compound and the thrombotic compound. One of skill in the art can readily determine if soaking is sufficient for a particular anti-thrombotic compound to exert its effects, or if a combination of soaking and injection should be utilized to effect an anti-thrombotic effect in the presence of a thrombotic compound. One of skill in the art can also readily determine what dosages to utilize by conducting dose-dependent studies as described in the Examples. Such dose-dependent studies are also standard in the art. In this way, one of skill in the art can determine what the “effective amount” of a particular compound is. As used herein, an “effective amount” is the amount of a compound is meant a nontoxic but sufficient amount of the compound to provide the desired effect. One of skill in the art will understand that the exact amount required will vary. However, an appropriate “effective amount” may be determined by one of ordinary skill in the art using only routine experimentation.

The present invention provides a method of identifying a thrombotic compound comprising: a) contacting a transgenic zebrafish containing platelets that express a reporter protein, with a test compound; b) comparing the platelets in the zebrafish contacted with the test compound with the platelets of a transgenic zebrafish that was not contacted with the test compound; and c) determining the effect of the test compound on platelet aggregation, such that if platelet aggregation in the zebrafish contacted with the test compound is greater than platelet aggregation in the zebrafish that was not contacted with the test compound, the compound is a thrombotic compound.

The thrombotic compounds identified by the methods of the present invention can be utilized to treat thrombocytopenia associated with pregnancy, thrombotic thrombocytopenia purpura hemolytic uremic syndrome or immune thrombocytopenic purpura. These thrombotic compounds are also useful in sealing off blood vessels or other areas in which restricted blood flow may be necessary.

Compounds such as warfarin cause a bleeding syndrome in adult zebrafish (Jagadeeswaran and Sheehan, 1999). Therefore, warfarin can be used to induce bleeding in zebrafish with fluorescent platelets. Zebrafish pretreated with warfarin can be utilized in the methods of the present invention to screen for thrombotic compounds that induce thrombosis. Such thrombotic compounds can be utilized to treat bleeding disorders or diseases such as those described above as well as other conditions where restricted blood flow may be necessary.

The thrombotic compounds identified by the methods of the present invention can also be utilized in other in vitro assays, such as cell adhesion assays or platelet aggregation assays to study the effects of the compounds on human platelets. Furthermore, the thrombotic compounds can be utilized in other in vivo animal models of thrombosis or other disease states associated with thrombosis, such as a mouse model, a rat model, a rabbit model or a baboon model of thrombosis to study their therapeutic effect.

The test compounds used in the methods described herein can be, but are not limited to, chemicals, small molecules, drugs, antibodies, peptides and secreted proteins. Test compounds in the form of cDNAs can also be tested in the methods of the present invention. cDNAs can be injected into transgenic zebrafish embryos of the present invention in order to assess the effects of the proteins encoded by these cDNAs on platelet aggregation. Test compounds that potentially inhibit or prevent platelet aggregation can be added before, concurrently with a thrombotic compound or after addition of a thrombotic compound. Several known anti-thrombotic compounds can be utilized as controls to determine the extent of the reduction or prevention of platelet aggregation by test compounds. These include, warfarin, aspirin, heparin (Ex. Lovenox), GPIIIa/IIb inhibitors (ex. ReoPro, Aggrastat, Integrilin), and tissue plasminogen activators (ex. Activase, Retavase), and platelet aggregation inhibitor (ex. Plavix). One of skill in the art can then compare the anti-thrombotic effects of test compounds with the anti-thrombotic effects of known compounds.

In all of the methods of the present invention, zebrafish can be contacted with a test compound (chemicals, small molecules, drugs, antibodies, peptides and secreted proteins) and/or a thrombotic compound by soaking the zebrafish for about 0.25, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 hours in the test compound and/or the thrombotic compound. As shown in the Examples, the zebrafish can be soaked in a test compound prior to contacting the zebrafish with a thrombotic compound. A zebrafish of the present invention can also be soaked in both a test compound and a thrombotic compound simultaneously. Also, once a zebrafish of the present invention has been contacted with a thrombotic compound, the zebrafish can be soaked in the test compound. Utilizing these soaking methods, it is possible to contact a zebrafish with small molecules as well as larger molecules such as peptides, proteins and antibodies to determine their effects on thrombosis.

Also provided by the present invention is a method of identifying genes expressed in platelets comprising: a) constructing a zebrafish platelet cDNA library; and b) identifying platelet genes. The genes identified from the platelet cDNA library can be involved in platelet function and/or thrombosis. Construction of the library is accomplished by methods standard in the art as well as those set forth in the Examples. The identification of platelet specific genes from a library is also described in the Examples. Therefore, the present invention also provides a method of identifying platelet-specific genes comprising: a) constructing a zebrafish platelet cDNA library; and b) identifying platelet-specific genes. One of skill in the art can identify genes expressed in platelets as platelet-specific genes by performing in situ hybridization, Western blot, or other immunocytochemistry techniques known in the art.

Upon identification of platelet genes, one of skill in the art would know how to compare the zebrafish sequence with other sequences in available databases in order to identify a human homologue of a platelet zebrafish gene. One of skill in the art would also be able to identify other homologues such as a mouse homologue or a rat homologue. Alternatively, sequences from the platelet zebrafish gene can be utilized as probes to screen a human library and identify human homologs. The zebrafish sequences can also be utilized to screen other animal libraries, such as a mouse library or a rat library. Upon identification of a mouse, rat or other animal homologue, these sequences can be utilized to screen for a human homologue, either by searching available databases, or screening a human library.

Upon identification of a platelet gene, the present invention also contemplates knocking out or knocking down platelet genes in zebrafish in order to determine their role in platelet function. As used herein, a “knockout” can be a platelet gene knockdown or the platelet gene can be knocked out by a mutation such as, a point mutation, an insertion, a deletion, a frameshift, or a missense mutation by techniques known in the art. Other gene silencing techniques, such as, GripNAs (Active Motif, Carlsbad, Calif.) can also be utilized to knock out or knock down genes. Also, transgenic zebrafish of the present invention can be crossed with a mutant fish line to knock out the platelet gene. Such knockouts can also be effected by utilizing morpholino technology, as described in the Examples. The use of morpholinos in zebrafish has been described in U.S. patent Publication No. 20020078471 (Ekker et al., U.S. Ser. No. 09/918,242, published Jun. 20, 2002) and is hereby incorporated by this reference in its entirety for information regarding the use of morpholinos in zebrafish. As shown herein, in the Examples, morpholinos recognizing the GPIIb or P2Y₁₂ mRNA, were effective in reducing in vivo ADP-induced aggregation in zebrafish.

For example, a transgenic zebrafish of the present invention that expresses a reporter protein in platelets can also have a platelet gene knocked out. One of skill in the art would compare embryonic development of this fish with a transgenic zebrafish expressing a reporter protein in platelets, that does not have the platelet gene knocked out. If there is a difference in the characteristics of the platelets and their interactions, the gene that has been knocked out plays a role in normal platelet function. The differences observed can be in platelet aggregation, platelet adhesion, platelet circulation or any other function associated with platelets. Other differences can include a difference in the ability to induce thrombosis.

Thus, the present invention also provides a method of identifying a platelet gene that is involved in platelet function comprising: a) comparing the platelets of a transgenic zebrafish containing platelets that express a reporter protein, with the platelets of a transgenic zebrafish containing platelets that express a reporter protein and has a platelet gene knocked out; and b) determining the effect of the platelet gene knockout on platelet function such that if there is a difference between the platelets of the transgenic zebrafish containing platelets that express a reporter protein and the platelets of the transgenic zebrafish containing platelets that express a reporter protein and has a platelet gene knockout, the platelet gene is involved in platelet function.

Also provided by the present invention is a method of identifying a platelet gene as a target for an anti-thrombotic compound comprising: a) contacting a transgenic zebrafish containing aggregated platelets that express a reporter protein with an anti-thrombotic compound; b) contacting a transgenic zebrafish containing aggregated platelets that express a reporter protein and has a platelet gene knocked out with an anti-thrombotic compound; c) comparing the platelets of the transgenic zebrafish containing aggregated platelets that express a reporter protein with the platelets in the transgenic zebrafish containing aggregated platelets that express a reporter protein and has a platelet gene knocked out; and d) determining the effect of the anti-thrombotic compound on platelet aggregation, such that if platelet aggregation in the transgenic zebrafish containing aggregated platelets that express a reporter protein is less than platelet aggregation in the zebrafish containing aggregated platelets that express a reporter protein and has a platelet gene knocked out, the platelet gene is a target for the anti-thrombotic compound.

The present invention also provides a method of identifying a platelet gene as a target for a thrombotic compound comprising: a) contacting a transgenic zebrafish containing platelets that express a reporter protein with a thrombotic compound; b) contacting a transgenic zebrafish containing platelets that express a reporter protein and has a platelet gene knocked out with a thrombotic compound; c) comparing the platelets in the transgenic zebrafish containing aggregated platelets that express a reporter protein with the platelets in the transgenic zebrafish containing aggregated platelets that express a reporter protein and has a platelet gene knocked out; and d) determining the effect of the thrombotic compound on platelet aggregation, such that if platelet aggregation in the transgenic zebrafish containing platelets that express a reporter protein is greater than platelet aggregation in the zebrafish containing platelets that express a reporter protein and has a platelet gene knocked out, the platelet gene is a target for the thrombotic compound.

The zebrafish containing a platelet gene knockout can also be utilized in the method of the present invention in order to identify potential drug targets. These knockout zebrafish can be utilized in the methods described herein to assess the effects of thrombotics and anti-thrombotics.

Therefore, the present invention also provides a method of identifying an anti-thrombotic compound that affects platelet aggregation via a platelet gene comprising: a) contacting a transgenic zebrafish containing aggregated platelets that express a reporter protein with a test compound; b) contacting a transgenic zebrafish containing aggregated platelets that express a reporter protein and has a platelet gene knocked out with a test compound; c) comparing the platelets in the transgenic zebrafish containing aggregated platelets that express a reporter protein with the platelets in the transgenic zebrafish containing aggregated platelets that express a reporter protein and has a platelet gene knocked out; and d) determining the effect of the test compound on platelet aggregation, such that if platelet aggregation in the transgenic zebrafish containing aggregated platelets that express a reporter protein is less than platelet aggregation in the zebrafish containing aggregated platelets that express a reporter protein and has a platelet gene knocked out, the compound is an anti-thrombotic compound that affects platelet aggregation via the platelet gene that has been knocked out.

Also provided by the present invention is a method of identifying a thrombotic compound that affects platelet aggregation via a platelet gene comprising:a) contacting a transgenic zebrafish containing platelets that express a reporter protein with a test compound; b) contacting a transgenic zebrafish containing platelets that express a reporter protein and has a platelet gene knocked out with a test compound; c) comparing the platelets in the transgenic zebrafish containing aggregated platelets that express a reporter protein with the platelets in the transgenic zebrafish containing aggregated platelets that express a reporter protein and has a platelet gene knocked out; and d) determining the effect of the test compound on platelet aggregation, such that if platelet aggregation in the transgenic zebrafish containing platelets that express a reporter protein is greater than platelet aggregation in the zebrafish containing platelets that express a reporter protein and has a platelet gene knocked out, the compound is a thrombotic compound that affects platelet aggregation via the platelet gene that has been knocked out.

In one example, thrombosis can be induced in a transgenic zebrafish expressing a reporter protein in platelets and in a transgenic fish expressing a reporter protein in platelets and containing a platelet gene knockout. A test compound is then administered to both fish. Either fish can receive the test compound first. One of skill in the art would then compare the knockout zebrafish with the zebrafish expressing a reporter protein in platelets that does not have a platelet gene knockout. If a decrease in platelet aggregation is observed in the zebrafish expressing a reporter protein in platelets, that does not have a platelet gene knockout as compared to the knockout zebrafish, the test compound is an anti-thrombotic compound that affects platelet aggregation via the platelet gene that has been knocked out. The anti-thrombotic compound can be interfering with transcription of this gene, translation of a protein encoded by the platelet gene or it may be inhibiting the platelet protein's activity either by inhibiting its ability to interact with other proteins, or degrading the protein.

In another example, a thrombotic compound can be administered to a transgenic zebrafish expressing a reporter protein in platelets and to a transgenic fish expressing a reporter protein in platelets that contains a platelet gene knockout. If an increase in platelet aggregation is observed in the zebrafish expressing a reporter protein in platelets, that does not have a platelet gene knockout as compared to the knockout zebrafish, the gene that has been knocked out is involved in platelet aggregation.

The present invention also provides a method of identifying a platelet gene that is involved in platelet function comprising: a) comparing the platelets in a transgenic zebrafish containing platelets that express a reporter protein, with the platelets in a transgenic zebrafish containing platelets that express a reporter protein and overexpress the product of the platelet gene; b) determining the effect of the overexpression of the product of the platelet gene on platelet function such that if there is a difference between the platelets of the transgenic zebrafish containing platelets that express a reporter protein and the transgenic zebrafish containing platelets that express a reporter protein and has a platelet gene overexpressed, the platelet gene is involved in platelet function.

Upon overexpression, increases or decreases in platelet aggregation may be observed by one of skill in the art. For example, if overexpression leads to increased platelet aggregation, the skilled artisan can contact the zebrafish overexpressing a platelet protein with an anti-thrombotic compound to determine how effective the anti-thrombotic compound is in the presence of the overexpressed protein. One of skill in the art can also contact the zebrafish with a test compound in order to identify compounds that decrease platelet aggregation in the presence of the overexpressed platelet protein. The extent to which overexpression increases platelet aggregation can be compared with the platelet aggregation observed upon contacting a zebrafish that does not overexpress the platelet protein with a thrombotic compound. These comparisons allow one of skill in the art to determine which anti-thrombotic compound and which dosages should be utilized to cause an anti-thrombotic effect.

If overexpression of a platelet gene leads to increased platelet aggregation, this can increase the sensitivity of the methods of the present invention. For example, it is possible for one of skill in the art to utilize a zebrafish that exhibits increased platelet aggregation as a result of overexpressing a platelet gene, in order to decrease the concentration of a thrombotic compound necessary to effect platelet aggregation or to eliminate contacting the zebrafish with a thrombotic compound.

Alternatively, if upon overepression, a decrease in platelet aggregation is observed, the skilled artisan can contact the zebrafish overexpressing a platelet protein with a thrombotic compound to determine how effective the thrombotic compound is in the presence of the overexpressed protein. One of skill in the art can also contact the zebrafish with test compound in order to identify compounds that increase platelet aggregation in the presence of the overexpressed platelet protein. The extent to which overexpression decreases platelet aggregation can be compared with the platelet aggregation observed upon contacting a zebrafish that does not overexpress the platelet protein with a known compound that decreases platelet aggregation. These comparisons allow one of skill in the art to determine which thrombotic compound and which dosages should be utilized to cause a thrombotic effect in the presence of the overexpressed protein.

The methods of the present invention can also be utilized to screen known compounds for their effects on platelet aggregation. For example, it is known that motapizone is a PDE (phosphodiesterase) III inhibitor. However motapizone has not been characterized as an anti-thrombotic compound that decreases platelet aggregation. By utilizing the methods of the present invention, applicants have shown that motapizone decreases platelet aggregation. Therefore, motapizone and its target can be useful for thrombosis therapy.

Therefore, the present invention also provides a method of identifying a known compound with a known target, as a compound that prevents thrombosis comprising: a) contacting a transgenic zebrafish expressing a reporter protein in platelets, with a thrombotic compound and a known compound; b) comparing the platelets in the zebrafish contacted with the thrombotic compound and the known compound with the platelets of a transgenic zebrafish that was contacted only with the thrombotic compound; and c) determining the effect of the known compound on the platelets, such that if platelet aggregation in the zebrafish contacted with the thrombotic compound and the known compound is less than platelet aggregation in the zebrafish that was contacted only with the thrombotic compound, the known compound is a compound that prevents thrombosis via its known target.

Also provided by the present invention is a method of identifying a known compound with a known target, as a thrombotic compound comprising: a) contacting a transgenic zebrafish containing platelets that express a reporter protein, with a known compound; b) comparing the platelets in the zebrafish contacted with the known compound with the platelets of a transgenic zebrafish that was not contacted with the known compound; and c) determining the effect of the test compound on platelet aggregation, such that if platelet aggregation in the zebrafish contacted with the known compound is greater than platelet aggregation in the zebrafish that was not contacted with the test compound, the known compound is a thrombotic compound that affects platelet aggregation via its known target.

Further provided by the present invention is a method of identifying a target for an anti-thrombotic compound comprising: a) contacting a transgenic zebrafish expressing a reporter protein in platelets, with a thrombotic compound and a known compound for which a target is known; b) comparing the platelets in the zebrafish contacted with the thrombotic compound and the known compound with the platelets of a transgenic zebrafish that was contacted only with the thrombotic compound; and c) determining the effect of the known compound on the platelets, such that if platelet aggregation in the zebrafish contacted with the thrombotic compound and the known compound is less than platelet aggregation in the zebrafish that was contacted only with the thrombotic compound, the known compound is a compound that prevents thrombosis via its known target, thus identifying a target for an anti-thrombotic compound.

The present invention also provides a method of identifying a target for a thrombotic compound comprising: a) contacting a transgenic zebrafish containing platelets that express a reporter protein, with a known compound for which a target is known; b) comparing the platelets in the zebrafish contacted with the known compound with the platelets of a transgenic zebrafish that was not contacted with the known compound; and c) determining the effect of the test compound on platelet aggregation, such that if platelet aggregation in the zebrafish contacted with the known compound is greater than platelet aggregation in the zebrafish that was not contacted with the test compound, the known compound is a thrombotic compound that affects platelet aggregation via its known target, thus identifying a target for a thrombotic compound.

The present invention is more particularly described in the following examples which are intended as illustrative only since numerous modifications and variations therein will be apparent to those skilled in the art.

### EXAMPLE I

Creation of Stable Megakaryocyte/Platelet Specific Fluorescent Fish

Several human genes are known to be expressed specifically in megakaryocytes. These include genes encoding the platelet receptor glycoprotein IIb (Poncz, et al., 1987), glycoprotein VI (Miura, et al, 2000) and the platelet ADP receptor P2Y₁₂ (Hollopeter, et al., 2001). The latter gene is also expressed in the brain, but is predominantly expressed in megakaryocytes. The zebrafish homologues of one or more of these genes can be isolated, as well as their corresponding promoters. Other promoters that can be utilized include, but are not limited to, the zebrafish homologues of the glycoprotein IX promoter, the glycoprotein Ib alpha promoter, the glycoprotein Ib beta promoter, the platelet factor IV promoter, the platelet basic protein promoter or the thrombopoietin receptor promoter. Since zebrafish hematopoiesis takes place in the kidney, kidney cDNA libraries provide a source of cloning for these genes. Genomic DNA can be isolated from a zebrafish P1-derived artificial chromosome (PAC) library using cDNA sequences as probes.

Zebrafish promoters are then fused to a vector containing sequences encoding a fluorescent protein, such as green fluorescent protein (GFP), enhanced green fluorescent protein (eGFP) or reef coral fluorescent protein (RCFP). One skilled in the art can use fluorescent protein genes isolated from a species of Anthozoa, developed by Clontech (Matz, et al., 1999). Linearized DNA fragments containing both promoter sequences and fluorescent protein sequences are injected into zebrafish embryos at the one cell stage. Two days after microinjection, embryos or larvae are observed for the presence of circulating fluorescent platelets. Embryos or larvae that have fluorescent platelets are then raised to adulthood. Adults are mated and screened for stable incorporation of the transgene, by microscopic examination of their progeny. Heterozygous carriers are raised to adulthood and intercrossed to obtain homozygous stocks of transgenic fish that express a fluorescent protein in platelets. This method is well-established in the laboratory of Dr. Shuo Lin (Long et al., 1997).

RNA Isolation

Total RNA is extracted from FACS-purified cells using the TRIzol RNA Isolation Kit (LIFE TECHNOLOGIES, Grand Island, N.Y.) and mRNA is isolated from the total RNA using PolyATtract System 1000 (Promega, Madison, Wis.). The protocols provided by LIFE TECHNOLOGIES and Promega are utilized for isolation of mRNA. At least 50 ng of mRNA will be prepared for cDNA library construction.

cDNA Library Construction

The SMART cDNA Library Construction Kit (Clontech), which was developed for constructing high-quality cDNA libraries from small quantities of RNA is utilized. As discussed above, although either total or poly A+ RNA may be used as a template for SMART cDNA synthesis, mRNA is utilized for the purposes of the present invention.

First-Strand cDNA is synthesized using 25 ng poly A+ mRNA isolated from GFP-positive cells. SMART/5′ oligonucleotide III and CDS/3′ oligonucleotide III is used in the MMLV reverse transcriptase reaction. The SMART/5′ oligonucleotide III contains an Sfi I site with AAT whereas the CDS/3′ oligonucleotide III contains an Sfi I site with GGC. This variation of AAT and GGC is used because Sfi I recognizes 5′GGCCNNNNNGGCC3′.

Low cycle, long-distance PCR (LD-PCR) is used to amplify the first-strand cDNA. KlenTaq Polymerase, a new 5′ PCR primer complementary to the SMART/5′ oligonucleotide III, and the CDS/3′ oligonucleotide III are used in the reaction. Currently, it is possible to amplify enough PCR products for library construction after 10 cycles. After amplification, a sample of the PCR product is analyzed with 1-kb DNA ladder size markers to determine the size and amount of PCR product.

As mentioned above, SMART oligonucleotide III and CDS oligonucleotide III contain Sfi I restriction sites. PCR products are digested with Sfi I restriction enzyme. This digestion generates DNA fragments with 5′ AAT and 3′ GGC overhangs. Digested products are then size-fractionated. Two cDNA pools are collected: one is 1-2 kb and another one is larger than 2 kb. After purification, the size-fractionated, Sfi I-digested cDNA is ligated to the dephosphorylated and Sfi I digested lambda TriplEx vector. One of these arms has a Sfi I site with TTA whereas the other one has a SfiI with CCG. Therefore, the cDNA inserts are cloned into the phage arms with their 5′ ends at the TTA arms and the 3′ ends at the CCG arms. The ligated products are packaged and a small portion of it plated out on LB plates for titering. 1-2×10⁶ independent clones are usually obtained. If the titer is as expected, remaining phages are converted into plasmid, to simplify sequencing and subtraction, as described below.

Library Characterization

First, 1,000 random clones from the library are sequences. This provides insight into the quality of the library, including the level of redundancy. Plasmid DNA obtained from the first 1,000 clones are used as driver to subtract redundant clones from the rest of the library. Normalization and subtraction is done according to Bonaldo, et al. (1996). Clones are sequenced until it is decided that all potential expressed sequences from the platelet library have been identified.

To determine whether novel zebrafish genes are appropriate drug targets, bioinformatics is utilized to establish whether human homologues exist. Second, whole mount in situ hybridization is performed to establish platelet specificity. Finally, functional information is obtained by knock-out technology, such as morpholinos (Nasevicius and Ekker, 2000) and grip NAs (Active Motif, Carlsbad, Calif.). Transient over-expression and over-expression of dominant negative constructs, when appropriate, is also used to provide functional information.

Assay Development

The transgenic platelet-specific fluorescent fish provides a novel way to visualize thrombotic processes in vivo, in a whole organism. By two days past fertilization, platelets should begin to circulate in the embryo. At this point in development, compounds are introduced that should cause platelet aggregation, such as thrombin, collagen or ADP. The effects of these thrombotic compounds are examined by direct observation of fluorescent platelets following injection. Small molecules are introduced to the embryo/larvae by soaking them in the compound, by injection or by allowing them to swallow the compounds after they have reached the age of 5 days past fertilization (dpf). Any combination of these administration methods can also be employed. For example, an embryo can be soaked in a compound followed by an injection of the compound. The clotting effect will be apparent as a change in the local concentration or distribution of fluorescence.

Zebrafish embryos can also be pretreated with anti-thrombotics, such as warfarin, aspirin, heparins (including low molecular weight heparins, such as Lovenox), GPIIIa/IIb inhibitors (ex. ReoPro, Aggrastat, Integrilin), and tissue plasminogen activators (ex. Activase, Retavase), platelet aggregation inhibitor (ex. Plavix) prior to the application of thrombotic compounds. For example, the platelet-specific zebrafish of the present invention can be soaked in the anti-thrombotic compound prior to the administration of the thrombotic compound. The zebrafish can also be soaked in the anti-thrombotic compound followed by an injection of the anti-thrombotic compound prior to administration of the thrombotic compound. One of skill in the art can determine the soaking time required for each compound as well as whether or not a combination of soaking and injection is necessary for a particular compound to exert its effects.

These assays can be used for target validation. Target genes, identified from the platelet library, will be overexpressed or knocked out/down in transgenic embryos or larvae, as described above. The resulting embryos/larvae will be subjected to the assays, so that any changes in response to thrombotics or anti-thrombotics can be observed.

High Throughput Screening

Several companies such as Imaging Resouces Incorporated, ATTO Bioscience and Union Biometrica, Inc. of Somerville, Mass. make fluorescent imagers/cell sorters designed for quantification of fluorescence in a three-dimensional organism. These imagers or any other imager, such as those produced by Perkin Elmer, sensitive enough to detect differences in fluorescence that result from thrombotic or anti-thrombotic molecule application can be utilized to establish a high throughput screen. In this high throughput screening method, embryos will be arrayed in 96 well plates in water or solutions of thrombotic compounds, test compounds or both, as described above. For guidance on the screening of small molecules, please see Peterson et al. (“Small molecule developmental screens reveal the logic and timing of vertebrate development” Proc. Natl. Acad. Sci. USA 97, 12965-12969 (2000)), which is hereby incorporated in its entirety by this reference.

At set time points, embryos will be collected and scanned for fluorescence. Changes in the local concentration and distribution of fluorescence can be observed as an indicator of clot formation or dissolution

In addition to visualizing whole embryos or larvae, embryos or larvae can be sonicated to break up into clumps of cells which will settle to the bottom of the culture plate. Thus, a traditional fluorescent plate scanner could be used to monitor fluorescence. Another possibility is to use suction to draw embryos onto a wet nitrocellulose filter and quantify fluorescence by scanning with a phosphoimager, such as the Storm phosphoimager.

Study of Platelets In Vitro

All of the methods of the present invention can be utilized in conjunction with in vitro assays to assess platelet function. It is possible to isolate fluorescent platelets from whole zebrafish blood to conduct in vitro assays, similar to those described in the literature (Jagadeeswaran and Sheehan, 1999; Jagadeeswaran, et al., 1999).

### EXAMPLE II

Materials

Reopro (abciximab), Aggrastat (tirofiban), Integrilin (eptifibatide), motapizone and clopidogrel were acquired from Aventis. Integrilin (solid form) was also attained from Millenium. Thrombin, ADP, Ticlopidine, hirudin, rat tail collagen type I and aspirin were from Sigma. Collagen horm was purchased from Nycomed. All other chemicals were reagent grade from Sigma.

Zebrafish

All zebrafish were maintained at 27° C. in re-circulating bio-filtered tanks. Wild-type Tübingen strain zebrafish were used for injecting linearized DNA containing the GPIIb-promoter and G-RCFP sequence. A stable zebrafish line expressing the enhanced green fluorescent protein (eGFP) in platelets was acquired from Dr. Robert Handin. Only GFP homozygous zebrafish were used for thrombotic and anti-thrombotic assays.

Creation of Z-Tag TG(GPIIb-G-RCFP) Zebrafish Line

The GPIIb promoter (provided by Dr. Robert Handin) was subcloned into pZsGreen-N1 expression vector (Clontech). The GPIIb:G-RCFP construct, containing the GPIIb promoter, GRCFP and poly-adenylation signal, was linearized and gel extracted. The purified DNA (40-60 ng) was injected into fertilized wild-type Tübingen eggs at the single-cell stage. Injected larvae were examined at 4-5 dpf (days post fertilization) for the presence of circulating fluorescent platelets. Only those with fluorescent platelets [149 positive transient expression of TG(GPIIb:G-RCFP)] were chosen and grown into adulthood. By screening their offspring, several founder TG(GPIIb:G-RCFP) zebrafish were discovered from a single mating pair with wild-type zebrafish. All positive embryos (F′ generation) were saved and grown for future use.

Thrombosis and Anti-Thrombotic Assay

Only larvae homozygous for the GPIIb-eGFP transgene, were used for the assay. Soaking experiments with anti-thrombotic compounds were initiated when larvae were 5 dpf and injections with thrombotic agents were performed the next day. Injection of compounds into the heart cavity of larvae was performed using a glass-pulled capillary needle (˜15 μm in diameter at the tip) attached to a microinjector. Phenol red (0.2%) was routinely used for all injections to ensure accurate injection into the heart cavity. All larvae were anaesthetized with tricaine during the injection and returned to fresh fish water before analysis. Fluorescence was observed using a stereomicroscope with a GFP specific filter set.

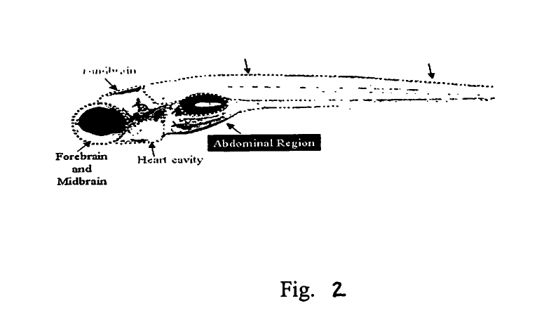

For some experiments (aspirin, Reopro, Integrilin, Ticlopidine and hirudin), larvae were scored according to the number of stationary platelets observed. The counts were done for sections of the larvae (FIG. 2) and later totaled for the larvae. For some experiments, anti-thrombotic compounds were considered successful when any moving platelets were observed in the presence of ADP. In these experiments (morpholino), control larvae (ADP alone) had no moving platelets.

Isolation of Platelets from Zebrafish Larvae

The larvae (6-7 dpf) were harvested and cell suspensions were prepared as previously described (Long et al., 1997). Briefly, anaesthetized larvae (approximately 1000) were crushed in a 1.5 ml eppendorf tube with a pestle. The cells were incubated with 0.05% Trypsin-EDTA for 15-20 min at 37° C. Thereafter, the cells were centrifuged at 1,000×g for 5 min and washed twice with PBS. The cells were placed at 4° C. overnight in Dulbecco's Modified Eagle Medium supplemented with 20% fetal bovine serum. The following day, cell suspension was poured through a 40 μm nylon filter and washed with PBS before being placed into the flow cytometer (University of Georgia flow cytometry core facility; Athens, Ga.). Cells were sorted using GFP channel or propidium iodide detector. GFP positive cells were collected in PBS and resorted to check for efficiency.

Morpholino Experiments

P2Y₁₂ (5′-AGCTGAGCTGCGTTGTTTGCTCCAT-3′) and GPIIb (5′-GACTGAATTCCAGTTTCTTGTCCAT-3′) morpholinos were purchased from Gene Tools (Philomath, Oreg.). Both of these morpholinos were designed to recognize the first 25 bases of coding sequence. Morpholino—(0.1 mM stock contained in 0.2% phenol red) or mock—(0.2% phenol red) injections were performed in one to four cell stage of homozygous TG(GPIIb:eGFP) zebrafish. These embryos were checked 2-4 hours after injection to eliminate non-fertilized or poorly developed eggs. The embryos were placed into Holtfretter's solution at 27° C. until used in the thrombosis assay (5-6 dpf). For the thrombosis assay, larvae were injected with 90 pmol of ADP, a concentration that immobilized all platelets in the larvae. A positive or negative effect was designated for each larva when there were moving or no moving platelets, respectively, after ADP challenge.

All data represented are expressed as means ± standard error. Statistical significance was determined when p<0.05 using student's unpaired, two-tailed distribution with unequal variance t-test for all samples.

Assay Development

Thrombotic and Anti-Thrombotic Assay Using ADP and Aspirin

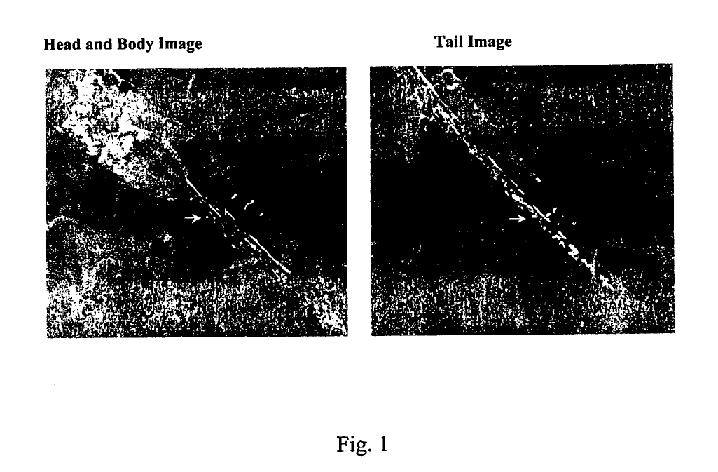

TG(GPIIb:GFP) zebrafish were bred to produce homozygous TG(GPIIb:eGFP) embryos. These embryos were grown to 5-7 days post fertilization (dpf; at this stage they are referred to as larvae) and were used to test several thrombotic and anti-thrombotic compounds. At 6 dpf, the larvae have developed a swim bladder and most (or all) of their yolk sac has disappeared. The fluorescent platelets may be visualized under a stereomicroscope (5-10×) using a mercury light source and GFP-specific excitation/emission filter set (FIG. 1).

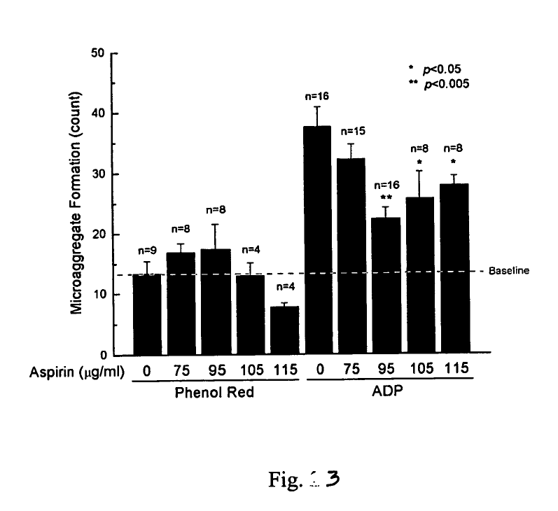

When 90 pmol of ADP was injected into the heart cavity of 6-7 dpf homozygous larvae, most or all of the platelets within the larvae stopped moving within 5 min of administration. This dose of ADP was not lethal as determined by the presence of a beating heart under bright light. The platelets were either single stagnant cells (labeled as microaggregates) or clumps of stagnant cells (macroaggregates). On average, there were about 38±3 microaggregates found in each of the ADP-injected larva (FIG. 3, n=16); this was statistically different from the control phenol red-injected larvae (13±2 microaggregates; n=9; p<0.001). The number of macroaggregates was not significant when compared between phenol red- and ADP-injected groups. The effect of ADP lasted for several hours (observed for up to 8 hours) and some of the larvae showed recovery by the next day. When 180 pmol of ADP was injected into the heart cavity of the larva, the larva died within 5-10 min (n=8). Lower concentrations of ADP did not induce a complete inhibition of platelet movement.

The effect of ADP-induced microaggregate formation was partially prevented when the larvae were soaked overnight with aspirin (95 and 105 μg/ml) before ADP challenge (FIG. 3). Aspirin alone had no adverse effect on platelet movement or the development of the larvae. At 115 μg/ml of aspirin, we observed a trend toward a decrease in microaggregate count within control phenol red-injected larvae. This reached statistical significance when the criterion for student t-test was less stringent, either using a paired distribution and/or using equal variance. At 125 μg/ml of aspirin, the larvae died from the overnight treatment.

Thrombin and Collagen as Agonists for Thrombosis

Two other pro-thrombotic compounds were tested in this in vivo zebrafish assay. First, injection of thrombin (3.38 ng, 0.018 NIH unit, n=15) induced platelet aggregation in zebrafish. Most of the platelets (approximately 85-90%) were arrested 5 min after injection of thrombin into the heart cavity. Unlike ADP, however, platelets began to move more freely within 25 min after thrombin injection, and within an hour, most platelets were mobile, similar to control phenol red-injected larvae. Nonetheless, thrombin consistently arrested platelet movement for 30 min.

Collagen horm (mostly type 1) from Nycomed was also utilized to induce thrombosis. Injection of 960 μg of collagen horm (from a 8 ng/μl stock in the injection needle) was able to induce about 80-90% microaggregate formation. A lower dose (240-480 pg) of the same stock concentration was ineffective at producing microaggregate formation. When higher stock concentrations (1 μg/μl or 100 ng/μl) of collagen horm were used for injections, the platelets were not responsive and showed no sign of microaggregation; therefore, no lethal dose of collagen was obtained. Applicants noted that collagen horm was very viscous and the substance did not diffuse readily into the tip of the microcapillary needle. This posed a technical problem and repeated experiments with collagen horm were not very successful. The use of epinephrine (18 pg) in conjunction with collagen did not enhance the effect of collagen on platelet aggregation. Due to the inconsistency experienced with collagen horm, anti-thrombotic assays using ADP were pursued.

Nycomed collagen horm did not yield consistent results in the in vivo thrombosis assay. Therefore, collagen type I that was purified from rat tail was tested. This collagen preparation had been previously shown to be effective in an in vitro platelet aggregation assay for zebrafish (Jagadeeswaran et al. 1999). When 360 ng of rat tail collagen type I was injected into the heart cavity of 6-7 dpf TG(GPIIb:eGFP) larvae, most or all of the platelets became immobile, suggesting the formation of aggregates. This effect lasted only for 3-5 min; thereafter, full recovery was observed. The effect was consistently seen in all larvae treated (n=32). A lethal dose with collagen type I was not obtained. Again, this may be due to the high viscosity of collagen type I, thus preventing a high concentration of the agonist into the 15 μm tip size of the capillary needle used for injection into the larva's heart cavity.

GPIIb/IIIa Antagonists: Reopro, Aggrastat and Integrilin

GPIIb/IIIa antagonists (Reopro, Aggrastat and Integrilin) have been successful in the clinical arena to prevent the formation of blood clots. These inhibitors are administered to patients via a bolus intravenous injection and are sometimes followed or preceded by a constant intravenous infusion for greater than 12 hours to prevent thrombotic events. These GPIIb/IIIa antagonists were tested in the zebrafish thrombosis assay.

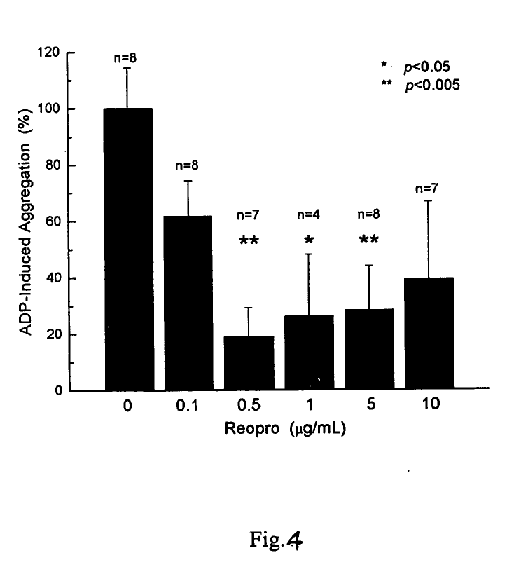

TG(GPIIb:eGFP) zebrafish larvae (5 dpf) were soaked in varying concentrations of Reopro (a human/murine chimeric antibody) overnight. The next day, larvae were injected with an additional 120 ng of Reopro 10 min before challenge with ADP. With this protocol, Reopro (0.5-5 μg/ml) dose-dependently inhibited platelet aggregation in live zebrafish larvae (FIG. 4). When less Reopro (60 ng) was injected following the overnight treatment, there was no effect of Reopro on the ADP-induced microaggregate formation. In addition, the injections of varying concentrations of Reopro (120 ng or less) alone without overnight soaking treatment did not antagonize the ADP-induced aggregation of platelets.

Aggrastat, a small molecule (soaking for 24 hours with 100 μg/ml followed by injection of 240 ng the next day for 10 min before ADP challenge), inhibited ADP-induced aggregation in TG(GPIIb:eGFP) larvae. Overnight treatment with lower concentrations (10-50 μg/ml) of Aggrastat was ineffective. Similar to Reopro, soaking alone or injection alone with Aggrastat did not antagonize the ADP-induced aggregate formation.

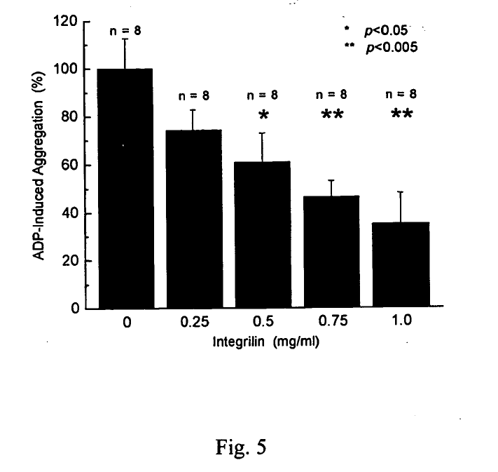

Integrilin, a cyclic peptide inhibitor of GPIIb/IIIa protein complex, reduced ADP-induced platelet aggregation in zebrafish, as shown in FIG. 5. When 90 pmol of ADP was injected into the heart cavity of 6-7 days post-fertilized (dpf) homozygous TG(GPIIb:eGFP) larvae, most or all of the platelets within the larvae stopped moving within 5 min of administration, due to the formation of platelet aggregates. This dose of ADP was not lethal as determined by the presence of a beating heart under bright field microscopy. When larvae were soaked in Integrilin overnight, a dose-dependent inhibition of ADP-induced aggregate formation was observed. At lower concentrations of Integrilin (0.25-1.0 mg/ml), there was no change in the number of platelet aggregates when compared to the control group, however at a higher concentration of Integrilin (5 mg/ml), all larvae died after the overnight treatment. The additional injection of Integrilin was not necessary for its antagonistic effect on ADP-induced aggregation. Unlike Reopro and Aggrastat, Integrilin was available as a powder, allowing a high soaking concentration to be performed. In summary, all three GPIIb/IIIa antagonists were effective as in vivo inhibitors of ADP-induced aggregate formation in zebrafish. This suggests that inhibitor's binding sites on the human GPIIb/IIIa receptor are shared by the zebrafish receptor.

Effect of Motapizone, a PDE III Inhibitor, on ADP-Induced Aggregation

An overnight soaking of 5 dpf larvae with motapizone (250 and 100 μM) was effective in blocking ADP-induced microaggregate formation. Partial inhibition of ADP-induced aggregation was observed with 50 μM motapizone while lower concentrations were ineffective. Injection of motapizone for 5 min before a challenge with ADP was ineffective at blocking the action of ADP on platelet aggregation. This suggested that the mechanism of ADP action on platelet aggregation in zebrafish is dependent on a decrease in cAMP levels, a phenomenon found in human platelets (Shulz et al., 1986a and 1986b). By inhibiting phosphodiesterase III, the breakdown of cAMP was inhibited, thereby ablating the ADP pro-thrombotic effect. ps Plavix, a P2Y₁₂ Receptor Antagonist, on ADP-Induced Aggregation

Clopidogrel (Plavix) has been shown to be an effective specific antagonist for P2Y₁₂ (Savi et al., 2001), a receptor that is found mainly in platelets and some regions of the brain (Hollopeter et al., 2001). In addition, Plavix is being used clinically to treat patients undergoing coronary stent placement (review in Shlansky-Goldberg, 2002). Currently, Plavix is ineffective for in vitro models because it needs to be metabolized by the liver to be effective (Savi et al., 1992; Savi et al., 2000). Therefore, the investigation of Plavix in zebrafish is of great relevance to demonstrate the value of an animal model for drug screening and to determine whether a similar ADP receptor exists in zebrafish.

When TG(GPIIb:GFP) zebrafish larvae were treated with Plavix (approximately 0.2-1 μg/ml) overnight, a partial inhibition of the ADP-induced aggregate formation was observed, suggesting the inhibition of a similar P2Y₁₂ receptor in zebrafish. The same concentration of Plavix was not effective in ablating aggregation induced by a high concentration of thrombin.

An exact Plavix concentration could not be determined because Plavix was provided as pills that were crushed into fine grains for the treatment. The fine grains of Plavix were not soluble in water, DMSO or glacial acetic acid. The mixture of 15 mg/ml of Plavix was used to make serial dilutions for overnight soakings. Lethal dose was found at 5 μg/ml and higher. However, even with the problem of Plavix's solubility, this data shows that Plavix is effective in zebrafish.

Anti-Thrombotic Assay with Ticlopidine and ADP

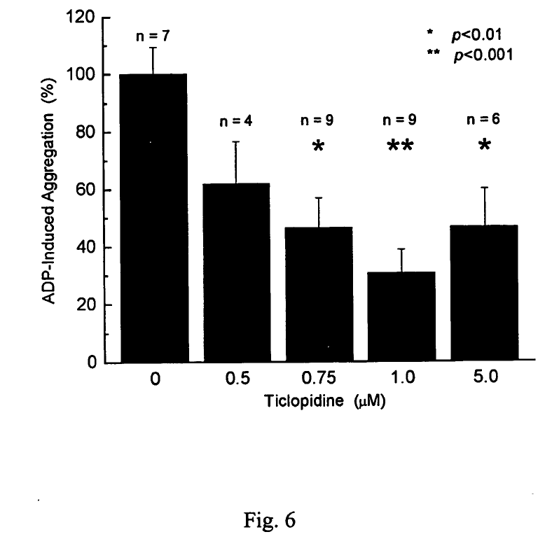

The inhibitory effect of Ticlopidine, a P2Y₁₂ receptor antagonist, on ADP-induced aggregation in zebrafish is illustrated in FIG. 6. When 5 dpf larvae were soaked in 0.5-5.0 μM Ticlopidine, a dose-dependent inhibition of ADP-induced platelet aggregation was observed (FIG. 6). At 10 μM Ticlopidine or higher, the larvae did not survive the overnight treatment. Thus, Ticlopidine can be metabolized by the larvae and exerts its effect on thrombosis in zebrafish in a manner similar to humans.

Anti-Thrombotic Assay with Hirudin and Thrombin/ADP

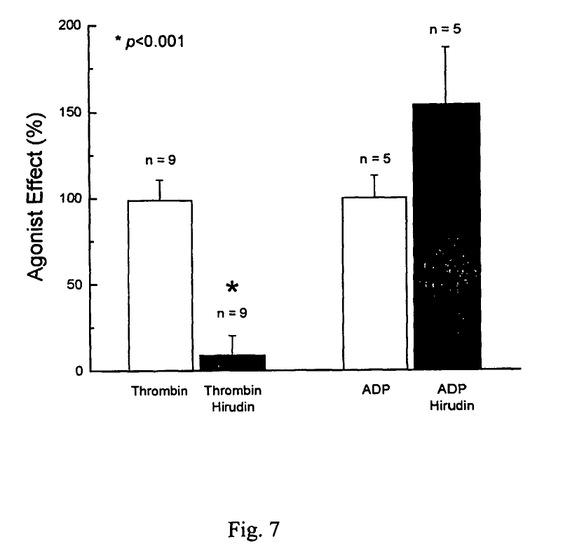

Since thrombin was another agonist that could be used in the in vivo thrombosis assay, whether or not a thrombin specific antagonist such as hirudin would be effective in zebrafish was determined. When thrombin (3.38 ng, 0.018 NIH unit) was injected into the heart cavity of 6 dpf larvae, most of the platelets were arrested within 5 min (FIG. 7). When hirudin (0.029 unit, where 1 unit of hirudin inactivates 1 NIH unit of thrombin) and thrombin were administered simultaneously to the larvae, no aggregation was observed. The same concentration of hirudin did not have any effect on ADP-induced aggregation. This showed that antagonists for thrombin itself can be validated or discovered using the in vivo thrombosis zebrafish assay.

Isolation of Platelets from TG(GPIIb:eGFP) Zebrafish

Platelets are one of the key players for thrombosis. When they are activated, the aggregation and clotting cascade ensues. Identifying novel platelet genes can assist in the development of drugs that will be advantageous for treating thrombosis in humans. Thus, creation of a platelet cDNA library would expedite the search for novel thrombosis targets.

Platelets in zebrafish are different from mammals and humans in that zebrafish platelets retain their nuclei. This suggests that transcripts and proteins are still being actively synthesized in zebrafish platelets. By isolating these platelets, a cDNA library from platelets can be made.

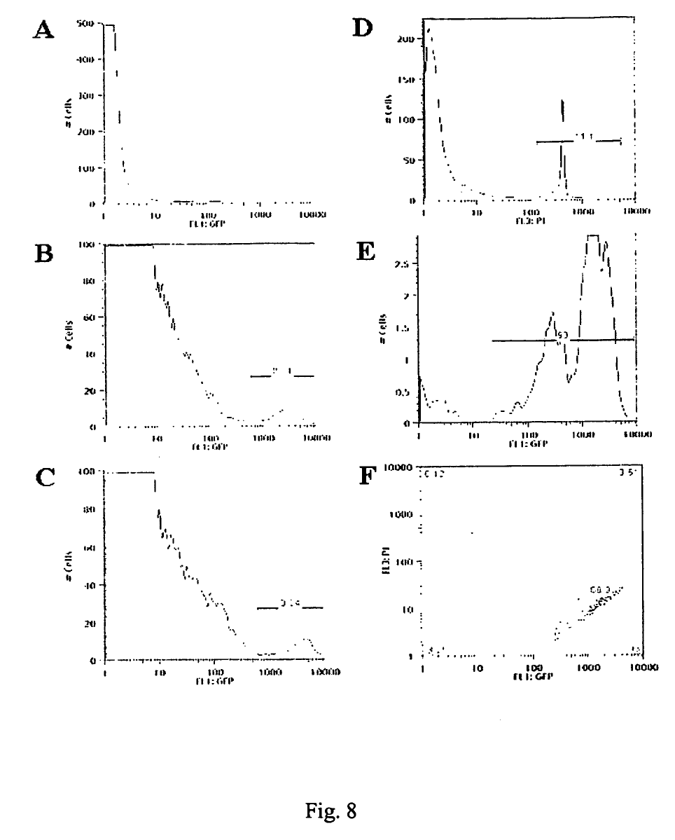

A round of platelet isolation from the TG(GPIIb:eGFP) zebrafish larvae was performed. Cells from wild-type larvae were sorted with the flow cytometer using the GFP channel. Very little fluorescence intensity was observed (FIG. 8a) for this cell population. When isolated cells from offsprings (6-7 dpf) of heterozygous zebrafish were sorted, there was a prominent peak of fluorescence near 2000 units, demonstrating the presence of GFP expressing cells. The percentage of cells that were within this fluorescent window was 0.21%. When isolated cells from offsprings of homozygous zebrafish were screened, there was a slightly higher peak near 5000 units, suggesting a higher amount of GFP expression within a given cell. The percentage of cells that were GFP positive from the homozygous larvae was 0.24%, a value slightly higher than the heterozygous larvae.

In addition to screening for GFP positive cells, cell viability after the isolation procedure was assessed. Propidium iodide was added to the cell suspension and cells were passed through the propidium iodide detector. In general, about 11.1% of the total cell population had taken up propidium iodide.

After collecting GFP positive cells from the first sort, the efficiency of GFP selection was assessed. Approximately 93% of the cells were GFP positive during the second sort. There were two prominent peaks, indicating the presence of the heterozygous- and homozygous-GFP expressing cells. When these cells were assessed for their viability, only 3.73% of the population had taken up propidium iodide. Thus, approximately 80.9-93.0% of the cells were GFP positive and viable. The total number of fluorescent cells collected was about 3×10⁴ from the 1000 larvae.

The percent of fluorescent cells isolated from the whole larvae was relatively low. Therefore, fluorescent cells were isolated by extracting blood from adult heterozygous TG(GPIIb:eGFP) zebrafish. From 3 adult zebrafish, approximately 2×10⁵ fluorescent cells were collected. This is much higher than the sample collected from larvae. From this initial sample of adult blood, it appears that approximately 200 adult zebrafish are necessary to collect 10⁷ platelets, an amount sufficient to make a cDNA library.

Detection of Platelet-Specific Genes Using In Situ Hybridization

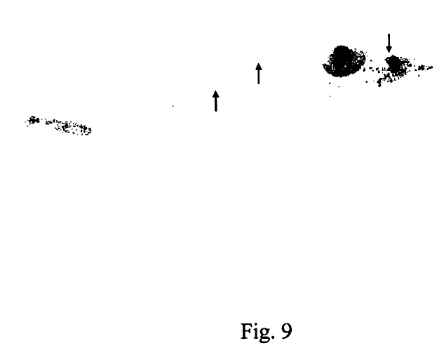

Genes isolated from the zebrafish's platelet cDNA library can be screened for their specificity to platelets by using whole mount in situ hybridization. An experiment was performed to ensure that such transcripts can be visualized in platelets using this technique. A 750 bp riboprobe for eGFP was made and used for in situ hybridization in 4 dpf and 8 dpf TG(GPIIb:eGFP) zebrafish. The riboprobe detected messenger RNA in the ventral trunk, a region where the intermediate cell mass is found (FIG. 9). The intermediate cell mass is where hematopoietic stem cells, such as red blood cells and platelets, originate during zebrafish development. In addition, single cell isolated staining was observed in the head region and throughout the trunk, suggesting the presence of platelets in circulation. For 8 dpf larvae, intense staining was observed in the pronephros, an area where platelets are produced during later developmental stages. At the same time, the intermediate cell mass is no longer present. Therefore, no intense staining was observed in the trunk region of the 8 dpf larvae, unlike at 4 dfp. In addition, single cell isolated staining was again observed throughout the head and trunk of the larvae, suggesting the presence of platelets in circulation.

Effect of Morpholinos in the In Vivo Thrombosis Assay

Knockout and/or knockdown experiments are very important for the understanding and validation of a protein's function in vivo. In the past, knockout experiments have been mostly done in mouse using targeted gene transfer methods. Creation of a chimeric mouse may take anywhere from 6 months to a year. Recent advances in antisense morpholino technology for zebrafish has shortened the time frame for studying gene knockout/knockdown to days and weeks.

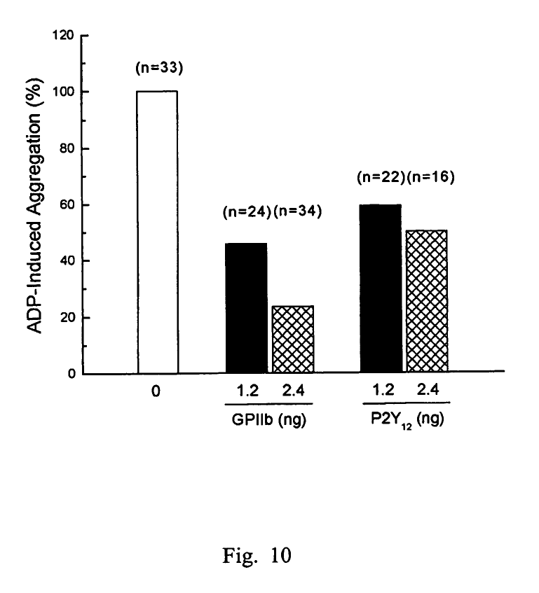

The present invention demonstrates that the use of morpholinos, against two proteins important for thrombosis, inhibited in vivo ADP-induced aggregation in zebrafish. When 90 pmol of ADP was injected into the heart cavity of homozygous TG(GPIIb:eGFP) larvae, a complete inhibition of platelet movement was observed. Since the phenotype was so dramatic, with most platelets moving in the absence of ADP and all platelets immobile in the presence of ADP, all larvae in morpholino experiments were scored for either presence or absence of moving platelets in the larvae after ADP administration. With 2.4 ng of GPIIb morpholino, 76.5% of the tested larvae (n=34) had moving platelets after ADP injection. This is a remarkable reduction when compared to the mock-injected (no morpholino) larvae where 100% of them had no moving platelets. In addition, the same amount of the P2Y₁₂ morpholino was 50% effective (FIG. 10). These experiments show that morpholinos are effective in the fluorescent thrombosis assay and therefore, novel genes for thrombosis can be discovered with morpholinos and the zebrafish thrombosis assays of the present invention.

Z-Tag Platelet-Specific Zebrafish Lines: Founders of TG(GPIIb:-GRCF) Zebrafish

Out of 42 zebrafish screened for stable integration of the GPIIb-GRCFP transgene, 4 founders were identified. Each of these founder lines has yielded heterozygous offspring that were raised and are being interbred to produce homozygous TG(GPIIb-GRCFP) zebrafish. For one line, there are over 80 zebrafish that are mixed heterozygous and homozygous. These can be screened to determine which are homozygous (about one-third). Thereafter, the homozygotes will be further interbred to produce a large population of homozygous zebrafish to create a platelet-specific cDNA library. Furthermore, the other 3 founders and their offsprings are being raised and are being amplified in a similar manner.

As stated above, in vitro experiments have shown that zebrafish platelets aggregate and coagulate in a manner similar to human platelets (Jagadeeswaran, et al., 1999; Sheehan et al. 2001). The present invention provides an in vivo assay for anti-thrombotic compounds in zebrafish, and furthermore, show that zebrafish respond to agonists and antagonists of thrombosis similar to humans. This was possible by labeling platelets in vivo with a green fluorescent protein. With such a zebrafish line, known antagonists and agonists for thrombosis were studied.

Agonists that are strong activators of platelets in humans were shown to have similar effects on platelets in zebrafish. The effect of ADP on zebrafish platelets was the most dramatic when compared to thrombin or collagen. ADP lasted much longer (hours) than either thrombin (tens of minutes) or collagen (5 min) and ADP immobilized all platelet movement within the larva, a result that was not usually seen with thrombin or collagen. Since ADP is commonly used for human platelet functional assays and was the most effective in zebrafish, further studies were performed using this assay. Furthermore, these results indicate that ADP, thrombin and possibly collagen receptors exist in zebrafish that are similar to receptors found in human.

Since GPIIb/IIIa receptors are known to play an integral role in thrombosis, their effect in a zebrafish anti-thrombotic assay was investigated. All three known antagonists for GPIIb/IIIa were effective in inhibiting ADP-induced aggregation. These three compounds have distinct mechanisms of action. Reopro is an antibody that binds irreversibly to a noncompetitive site of GPIIb/IIIa receptors. Both Aggrastat and Integrilin are reversible antagonists that bind competitively within the active site of GPIIb/IIIa receptors; however, their pharmacological structures are quite different: Aggrastat is a small synthetic molecule while Integrilin is a peptide. This suggests that the GPIIb/IIIa receptors expressed in zebrafish are similar in the regions where these drugs exert their effects on human GPIIb/IIIa receptors. The zebrafish GPIIb homologue shares approximately 45% identity and 65% similarity with the human protein. It is common for the active/binding sites within a family of proteins to be highly conserved; for example, between the zebrafish and human GPIIb proteins, therefore explaining the results of these distinct GPIIb antagonists in the in vivo zebrafish anti-thrombotic assay.

The mechanism for platelet activation in zebrafish was also addressed by using antagonists for intracellular second messengers. For instance, aspirin, a known cyclooxygenase antagonist, was effective at inhibiting the ADP-induced aggregation of platelets in zebrafish. This is most likely due to a reduction in thromboxane A₂ production, a stimulator of platelet aggregation. Motapizone was also effective in reducing platelet aggregation in zebrafish. This inhibitor blocks PDE III, an enzyme that metabolizes cAMP within a cell, resulting in an accumulation of cAMP. Platelet activation requires a reduction of cAMP level, especially when stimulated by ADP.

Antagonists of ADP receptors, such as ticlopidine and clopidogrel, have been effective in the clinical scenario. However, such antagonists cannot be tested in vitro, because they must be metabolized to be effective. In the in vivo zebrafish model of the present invention, Plavix was effective at inhibiting the ADP-induced platelet aggregation, suggesting that it was metabolized properly by the zebrafish. Plavix is known to be specific for the P2Y₁₂ receptor and does not have any effect on P2Y₁ or P2X₁ receptors, which are also expressed on human platelets. Therefore, it is likely that zebrafish platelets express a homologue of the P2Y₁₂ receptor. To this end, a homologue of the P2Y₁₂ receptor was identified. The zebrafish protein sequence is about 52% identical and 67% similar to the human sequence. The present invention therefore contemplates further studies with P2Y₁₂ receptor antagonists, such as ticlopidine, ARL 66096 and AR-C69931MX (Jung & Moroi, 2001; Storey, 2001) to demonstrate the importance of P2Y₁₂ receptors in zebrafish thrombosis. In addition, Gregory and Jagadeeswaran (2002) have suggested the expression of another ADP receptor, P2Y₁, in zebrafish. By utilizing specific antagonists for this receptor in the in vivo zebrafish model of the present invention, it is possible to understand the role of ADP receptors for thrombosis.

Applicants have shown that with more compounds tested in the in vivo platelet-specific fluorescent thrombosis assay, more evidence has accumulated that thrombotic events in zebrafish and humans are similar. Three different types of GPIIb/IIIa antagonists, Reopro, Integrilin and Aggrastat, have been demonstrated to be effective in the platelet-specific fluorescent thrombosis assay. In addition, applicants have shown that the ADP receptor, specifically P2Y₁₂ receptor, plays an integral role in zebrafish platelet aggregation, through the use of Ticlopidine and Plavix. The specificity of the anti-thrombotic compounds was also examined. For example, hirudin inhibited thrombin-induced aggregation, but not ADP-induced aggregation. This provides more evidence that the thrombosis events between zebrafish and humans are similar.

This pharmacological data suggested that zebrafish and human GPIIb/IIIa protein complexes were homologous. To further support these similarities, antisense technology using morpholinos was employed to study specific genes. In order to design these morpholinos, the 5′ untranslated region and the first 25 bp of the mRNA must be identified. This was accomplished by using 5′ RACE experiments for both GPIIb and P2Y₁₂. The GPIIb morpholino showed a more pronounced effect than the P2Y₁₂ morpholino. Therefore applicants have shown that 1) thrombotic genes such as P2Y₁₂ and GPIIb receptors were found in zebrafish and 2) the knockdown of the corresponding proteins with morpholinos was effective in reducing agonist-induced platelet aggregation. This provides a new tool to identify novel thrombosis genes that may ultimately lead to an ideal drug target for therapy.

Thus, the present invention shows that there is tremendous value in the use of morpholinos and the zebrafish technology of the present invention to identify and validate targets for thrombosis. This can be accomplished by creating a cDNA library to identify platelet genes. Applicants have shown that platelets can be readily purified from Z-Tag larvae and/or adults. Since about 10⁷ platelets will be required to make a cDNA library, a few hundred adult zebrafish are necessary to purify enough platelets for the cDNA library. Furthermore, to assist with narrowing the pool of genes that may be screened in the thrombosis assay, applicants have also shown that in situ hybridization is a reliable tool to distinguish platelet-specific gene expression. Thus, the present invention also provides the detection of other genes via in situ hybridization in zebrafish, such as P2Y₁₂, P2Y₁ (another ADP receptor), GPIIb and PAR-1 (thrombin receptor) receptors, to show platelet specificity.

Throughout this application, various publications are referenced. The disclosures of these publications in their entireties are hereby incorporated by reference into this application in order to more fully describe the state of the art to which this invention pertain.

### References

Bonaldo M F, Lennon G, Soares M B. (1996). Normalization and subtraction: two approaches to facilitate gene discovery. Genome Res. 6: 791-806
Brownlie A, Donovan A, Pratt S J, Paw B H, Oates A C, Brugnara C, Witkowska H E, Sassa S, Zon L I. (1998). Positional cloning of the zebrafish sauternes gene: a model for congenital sideroblastic anaemia. Nat Genet. 20: 244-50.
Childs S, Weinstein B M, Mohideen M A, Donohue S, Bonkovsky H, Fishman M C. (2000). Zebrafish dracula encodes ferrochelatase and its mutation provides a model for erythropoietic protoporphyria. Curr. Biol. 10: 1001-4.
Donovan A, Brownlie A, Zhou Y, Shepard J, Pratt S J, Moynihan J, Paw B H, Drejer A, Barut B, Zapata A, Law T C, Brugnara C, Lux S E, Pinkus G S, Pinkus J L, Kingsley P D, Palis J, Fleming M D, Andrews N C, Zon L I. (2000). Positional cloning of zebrafish ferroportinI identifies a conserved vertebrate iron exporter. Nature. 403: 776-81.
Gregory, M. & Jagadeeswaran, P. (2002). Selective labeling of zebrafish thrombocytes: quantification of thrombocyte function and detection during development. Blood Cells, Molecules, and Diseases 28: 418-427.
Herrmann K S. (1983). Platelet aggregation induced in the hamster cheek pouch by a photochemical process with excited fluorescein isothiocyanate-dextran. Microvasc. Res. 26: 238-249.

Hollopeter G, Jantzen H-M, Vincent D, Li G, England L, Ramakrishnan V, Yang R-B, Nurden P, Nurden A, Julius D, Conley P B. (2001). Identification of the platelet ADP receptor targeted by anti-thrombotic drugs. Nature 409: 202-207.
Jagadeeswaran P, Liu Y C. (1997a). Developmental expression of thrombin in zebrafish embryos: a novel model to study hemostasis. Blood Cells Mol Dis. 23: 147-56.
Jagadeeswaran P, Liu Y C. (1997b). A hemophilia model in zebrafish: analysis of hemostasis. Blood Cells Mol Dis. 23: 52-7.
Jagadeeswaran P, Liu Y C, Eddy C A. (1997). Effects of hirudin (thrombin specific inhibitor) in zebrafish embryos: a developmental role for thrombin. Blood Cells Mol Dis. 23:410-4.
Jagadeeswaran P, Sheehan J P. (1999). Analysis of blood coagulation in the zebrafish. Blood Cells Mol Dis. June-August 1999;25(3-4):239-49.
Jagadeeswaran P, Sheehan J P, Craig F E, Troyer D. (1999). Identification and characterization of zebrafish thrombocytes. Br J Haematol. 1999 107(4):731-8.
Jagadeeswaran P, Gregory M, Johnson S, Thankavel B. (2000a). Haemostatic screening and identification of zebrafish mutants with coagulation pathway defects: an approach to identifying novel haemostatic genes in man. Br J Haematol. 110: 946-56.
Jagadeeswaran P, Gregory M, Zhou Y, Zon L, Padmanabhan K, Hanumanthaiah R. (2000b). Characterization of zebrafish full-length prothrombin cDNA and linkage group mapping. Blood Cells Mol Dis. 26: 479-89.
Jessen J R, Willett C E, Lin S. (1999). Artificial chromosome transgenesis reveals long-distance negative regulation of rag1 in zebrafish. Nat Genet. 23:15-6.
Leadley R J, Chi L, Rebello S S, Gagnon A. (2000). Contribution of in vivo models of thrombosis to the discovery and development of novel anti-thrombotic agents. J. Pharm. Toxicol. Meth. 43: 101-116.
Liao E C, Paw B H, Peters L L, Zapata A, Pratt S J, Do C P, Lieschke G, Zon L I. (2000). Hereditary spherocytosis in zebrafish riesling illustrates evolution of erythroid beta-spectrin structure, and function in red cell morphogenesis and membrane stability. Development. 127: 5123-32.
Long Q, Meng A, Wang H, Jessen J R, Farrell M J, Lin S. (1997). GATA-1 expression pattern can be recapitulated in living transgenic zebrafish using GFP reporter gene. Development. 124:4105-11.
Matz M V, Fradkov A F, Labas Y A, Savitsky A P, Zaraisky A G, Markelov M L, Lukyanov S A. (1999) Fluorescent proteins from nonbioluminescent Anthozoa species. Nat. Biotechnol. 17: 969-73
Miura Y, Ohnuma M, Jung S M, Moroi M. (2000). Cloning and expression of the platelet-specific collagen receptor glycoprotein VI. Thromb. Res. 98: 301-309.
Nasevicius A, Ekker S C. (2000). Effective targeted gene ‘knockdown’ in zebrafish. Nat Genet. 26: 216-20.
Poncz M, Eisman R, Heidenreich R, Silver S M, Vilaire G, Surrey S, Schwartz E, Bennett J S. (1987) Structure of the platelet membrane glycoprotein IIb. Homology to the alpha subunits of the vitronectin and fibronectin membrane receptors. J. Biol. Chem. 262:8476-82.
Sato M and Ohshima N (1984). Platelet thrombus induced in vivo by filtered light and fluorescent dye in mesenteric microvessels of the rat. Thromb. Res. 35: 319-334.
Savi, P. Herbert, J. M., Pflieger, A. M., Dol, F., Delebassee, D., Combalbert, J., Defreyn, G. & Maffrand, J. P. (1992). Importance of hepatic metabolism in the antiaggregation activity of the thienopyridine clopidogrel. Biochemical Pharmacology 44: 527-532.
Savi, P., Pereillo, J. M., Uzabiaga, M. F., Combalbert, J., Picard, C., Maffrand, J. P., Pascal, M. & Herbert, J. M. (2000). Identification and biological activity of the active metabolic of clopidogrel. Thrombosis and Haemostasis 84: 891-896.
Savi, P., Labouret, C., Delesque, N., Guette, F., Lupker, J. & Herbert, J. M. (2001) P2Y12, a new platelet ADP receptor, target of clopidogrel. Biochemical and Biophysical Research Communications 283: 379-383.
Sheehan J, Templer M, Gregory M, Hanumanthaiah R, Troyer D, Phan T, Thankavel B, Jagadeeswran P. (2001). Demonstration of the extrinsic coagulation pathway in teleostei: Identification of zebrafish coagulation factor VII.
Shlansky-Goldberg, R. (2002). Platelet aggregation inhibitors for use in peripheral vascular interventions: what can we learn from the experience in the coronary arteries? Journal of Vascular Intervention Radiology 13:229-246.
Schulz, V., Fischer, W., Hanselle, U., Huhmann, W. & Zietsch, V. (1986a). Hypotensive and antiplatelet actions of motapizone depend on dose and time after ingestion. European Journal of Clinical Pharmacology 31: 405-410.
Schulz, V., Fischer, W., Hanselle, U., Huhmann, W. & Zietsch, V. (1986b). Inhibition of thrombocyte aggregation by oral motapiczone and other drugs. European Journal of Clinical Pharmacology 31: 411-414.
Wang H, Long Q, Marty S D, Sassa S, Lin S. (1998). A zebrafish model for hepatoerythropoietic porphyria. Nat. Genet. 20: 239-43.
Weinstein B M, Stemple D L, Driever W D, Fishman M C. (1995). Gridlock, a localized heritable vascular patterning defect in the zebrafish. Nat. Med. 11: 1143-1147.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of identifying an anti-thrombotic compound comprising:
a) contacting a transgenic zebrafish containing aggregated platelets that express a reporter protein, with a test compound;
b) comparing the platelets in the zebrafish contacted with the test compound with the platelets of a transgenic zebrafish that was not contacted with the test compound; and
c) determining the effect of the test compound on platelet aggregation, such that if platelet aggregation in the zebrafish contacted with the test compound is less than platelet aggregation in the zebrafish that was not contacted with the test compound, the compound is an anti-thrombotic compound.

2. The method of claim 1, wherein the reporter protein is a fluorescent protein.

3. The method of claim 2, wherein the fluorescent protein is green fluorescent protein.

4. The method of claim 2, wherein the fluorescent protein is reef coral fluorescent protein.

5. A method of identifying a compound that prevents thrombosis comprising:
a) contacting a transgenic zebrafish expressing a reporter protein in platelets, with a thrombotic compound and test compound,
b) comparing the platelets in the zebrafish contacted with the thrombotic compound and the test compound with the platelets of a transgenic zebrafish that was contacted only with the thrombotic compound; and
c) determining the effect of the test compound on the platelets, such that if platelet aggregation in the zebrafish contacted with the thrombotic compound and the test compound is less than platelet aggregation in the zebrafish that was contacted only with the thrombotic compound, the compound prevents thrombosis.

6. The method of claim 5, wherein the reporter protein is a fluorescent protein.

7. The method of claim 6, wherein the fluorescent protein is green fluorescent protein.

8. The method of claim 6, wherein the fluorescent protein is reef coral fluorescent protein.

9. A method of identifying a thrombotic compound comprising:
a) contacting a transgenic zebrafish containing platelets that express a reporter protein, with a test compound;
b) comparing the platelets in the zebrafish contacted with the test compound with the platelets of a transgenic zebrafish that was not contacted with the test compound; and
c) determining the effect of the test compound on platelet aggregation, such that if platelet aggregation in the zebrafish contacted with the test compound is greater than platelet aggregation in the zebrafish that was not contacted with the test compound, the compound is a thrombotic compound.

10. The method of claim 9, wherein the reporter protein is a fluorescent protein.

11. The method of claim 10, wherein the fluorescent protein is green fluorescent protein.

12. The method of claim 10, wherein the fluorescent protein is reef coral fluorescent protein.

13. A method of identifying a platelet gene that is involved in platelet function comprising:
a) comparing the platelets in a transgenic zebrafish containing platelets that express a reporter protein, with the platelets in a transgenic zebrafish containing platelets that express a reporter protein and has a platelet gene knocked out; and
b) determining the effect of the platelet gene knockout on platelet function such that if there is a difference between the platelets of the transgenic zebrafish containing platelets that express a reporter protein and the transgenic zebrafish containing platelets that express a reporter protein and has a platelet gene knockout, the platelet gene is involved in platelet function.

14. The method of claim 13, wherein the reporter protein is a fluorescent protein.

15. The method of claim 14, wherein the fluorescent protein is green fluorescent protein.

16. The method of claim 14, wherein the fluorescent protein is coral reef fluorescent protein.

17. A method of identifying a platelet gene as a target for an anti-thrombotic compound comprising:
a) contacting a transgenic zebrafish containing aggregated platelets that express a reporter protein with an anti-thrombotic compound;
b) contacting a transgenic zebrafish containing aggregated platelets that express a reporter protein and has a platelet gene knocked out with an anti-thrombotic compound;
c) comparing the platelets in the transgenic zebrafish containing aggregated platelets that express a reporter protein with the platelets in the transgenic zebrafish containing aggregated platelets that express a reporter protein and has a platelet gene knocked out; and
d) determining the effect of the anti-thrombotic compound on platelet aggregation, such that if platelet aggregation in the transgenic zebrafish containing aggregated platelets that express a reporter protein is less than platelet aggregation in the zebrafish containing aggregated platelets that express a reporter protein and has a platelet gene knocked out, the platelet gene is a target for the anti-thrombotic compound.

18. The method of claim 17, wherein the reporter protein is a fluorescent protein.

19. The method of claim 18, wherein the fluorescent protein is green fluorescent protein.

20. The method of claim 18, wherein the fluorescent protein is reef coral fluorescent protein.

21. A method of identifying a platelet gene as a target for a thrombotic compound comprising
a) contacting a transgenic zebrafish containing platelets that express a reporter protein with a thrombotic compound;
b) contacting a transgenic zebrafish containing platelets that express a reporter protein and has a platelet gene knocked out with a thrombotic compound;
c) comparing the platelets in the transgenic zebrafish containing aggregated platelets that express a reporter protein with the platelets in the transgenic zebrafish containing aggregated platelets that express a reporter protein and has a platelet gene knocked out; and
d) determining the effect of the thrombotic compound on platelet aggregation, such that if platelet aggregation in the transgenic zebrafish containing platelets that express a reporter protein is greater than platelet aggregation in the zebrafish containing platelets that express a reporter protein and has a platelet gene knocked out, the platelet gene is a target for the thrombotic compound.

22. The method of claim 21, wherein the reporter protein is a fluorescent protein.

23. The method of claim 22, wherein the fluorescent protein is green fluorescent protein.

24. The method of claim 22, wherein the fluorescent protein is coral reef fluorescent protein.

25. A method of identifying an anti-thrombotic compound that affects platelet aggregation via a platelet gene comprising:
a) contacting a transgenic zebrafish containing aggregated platelets that express a reporter protein with a test compound;
b) contacting a transgenic zebrafish containing aggregated platelets that express a reporter protein and has a platelet gene knocked out with a test compound;
c) comparing the platelets in the transgenic zebrafish containing aggregated platelets that express a reporter protein with the platelets in the transgenic zebrafish containing aggregated platelets that express a reporter protein and has a platelet gene knocked out;
d) determining the effect of the test compound on platelet aggregation, such that if platelet aggregation in the transgenic zebrafish containing aggregated platelets that express a reporter protein is less than platelet aggregation in the zebrafish containing aggregated platelets that express a reporter protein and has a platelet gene knocked out, the compound is an anti-thrombotic compound that affects platelet aggregation via the platelet gene that has been knocked out.

26. The method of claim 25, wherein the reporter protein is a fluorescent protein.

27. The method of claim 26, wherein the fluorescent protein is green fluorescent protein.

28. The method of claim 26, wherein the fluorescent protein is green fluorescent protein.

29. A method of identifying a thrombotic compound that affects platelet aggregation via a platelet gene comprising:
a) contacting a transgenic zebrafish containing platelets that express a reporter protein with a test compound;
b) contacting a transgenic zebrafish containing platelets that express a reporter protein and has a platelet gene knocked out with a test compound;
c) comparing the platelets in the transgenic zebrafish containing aggregated platelets that express a reporter protein with the platelets in the transgenic zebrafish containing aggregated platelets that express a reporter protein and has a platelet gene knocked out,
d) determining the effect of the test compound on platelet aggregation, such that if platelet aggregation in the transgenic zebrafish containing platelets that express a reporter protein is greater than platelet aggregation in the zebrafish containing platelets that express a reporter protein and has a platelet gene knocked out, the compound is a thrombotic compound that affects platelet aggregation via the platelet gene that has been knocked out.

30. The method of claim 29, wherein the reporter protein is a fluorescent protein.

31. The method of claim 30, wherein the fluorescent protein is green fluorescent protein.

32. The method of claim 30, wherein the fluorescent protein is reef coral fluorescent protein.

33. A method of identifying platelet-specific genes comprising:
a) constructing a zebrafish platelet cDNA library; and
b) identifying a platelet specific gene.

34. The method of claim 33, wherein the platelet specific gene is identified via ill situ hybridization.

35. The method of claim 33, further comprising identifying homologues of the zebrafish platelet-specific genes.

36. The method of claim 35, wherein the homologue is a human homologue.

37. The method of claim 35, wherein the homologue is a mouse homologue.

38. The method of claim 35, wherein the homologue is a rat homologue.

39. The present invention also provides a method of identifying a platelet gene that is involved in platelet function comprising:
a) comparing the platelets in a transgenic zebrafish containing platelets that express a reporter protein, with the platelets in a transgenic zebrafish containing platelets that express a reporter protein and overexpress the product of the platelet gene; and
b) determining the effect of the overexpression of the product of the platelet gene on platelet function such that if there is a difference between the platelets of the transgenic zebrafish containing platelets that express a reporter protein and the transgenic zebrafish containing platelets that express a reporter protein and has a platelet gene overexpressed, the platelet gene is involved in platelet function.

40. The method of claim 39, wherein the reporter protein is a fluorescent protein.

41. The method of claim 40, wherein the fluorescent protein is green fluorescent protein.

42. The method of claim 40, wherein the fluorescent protein is reef coral fluorescent protein.

43. A method of identifying a known compound with a known target, as a compound that prevents thrombosis comprising:
a) contacting a transgenic zebrafish expressing a reporter protein in platelets, with a thrombotic compound and the known compound;
b) comparing the platelets in the zebrafish contacted with the thrombotic compound and the known compound with the platelets of a transgenic zebrafish that was contacted only with the thrombotic compound;
c) determining the effect of the known compound on the platelets, such that if platelet aggregation in the zebrafish contacted with the thrombotic compound and the known compound is less than platelet aggregation in the zebrafish that was contacted only with the thrombotic compound, the known compound is a compound that prevents thrombosis via its known target.

44. The method of claim 43, wherein the reporter protein is a fluorescent protein.

45. The method of claim 44, wherein the fluorescent protein is green fluorescent protein.

46. The method of claim 44, wherein the fluorescent protein is reef coral fluorescent protein.

47. A method of identifying a known compound with a known target, as a thrombotic compound comprising:
a) contacting a transgenic zebrafish containing platelets that express a reporter protein, with a known compound;
b) comparing the platelets in the zebrafish contacted with the known compound with the platelets of a transgenic zebrafish that was not contacted with the known compound; and
c) determining the effect of the test compound on platelet aggregation, such that if platelet aggregation in the zebrafish contacted with the known compound is greater than platelet aggregation in the zebrafish that was not contacted with the test compound, the known compound is a thrombotic compound that affects platelet aggregation via its known target.

48. The method of claim 47, wherein the reporter protein is a fluorescent protein.

49. The method of claim 48, wherein the fluorescent protein is green fluorescent protein.

50. The method of claim 48, wherein the fluorescent protein is reef coral fluorescent protein.

51. A method of identifying a target for an anti-thrombotic compound comprising:
a) contacting a transgenic zebrafish expressing a reporter protein in platelets, with a thrombotic compound and a known compound for which a target is known;
b) comparing the platelets in the zebrafish contacted with the thrombotic compound and the known compound with the platelets of a transgenic zebrafish that was contacted only with the thrombotic compound;
c) determining the effect of the known compound on the platelets, such that if platelet aggregation in the zebrafish contacted with the thrombotic compound and the known compound is less than platelet aggregation in the zebrafish that was contacted only with the thrombotic compound, the known compound is a compound that prevents thrombosis via its known target, thus identifying a target for an anti-thrombotic compound.

52. The method of claim 51, wherein the reporter protein is a fluorescent protein.

53. The method of claim 52, wherein the fluorescent protein is green fluorescent protein.

54. The method of claim 52, wherein the fluorescent protein is reef coral fluorescent protein.

55. A method of identifying a target for a thrombotic compound comprising:
a) contacting a transgenic zebrafish containing platelets that express a reporter protein, with a known compound for which a target is known;
b) comparing the platelets in the zebrafish contacted with the known compound with the platelets of a transgenic zebrafish that was not contacted with the known compound; and
c) determining the effect of the test compound on platelet aggregation, such that if platelet aggregation in the zebrafish contacted with the known compound is greater than platelet aggregation in the zebrafish that was not contacted with the test compound, the known compound is a thrombotic compound that affects platelet aggregation via its known target, thus identifying a target for a thrombotic compound.

56. The method of claim 55, wherein the reporter protein is a fluorescent protein.

57. The method of claim 56, wherein the fluorescent protein is green fluorescent protein.

58. The method of claim 56, wherein the fluorescent protein is reef coral fluorescent protein.
